SEQUENCE LISTING (5 sequences)

SEQ ID NO: 1 - protein, 149 aa, synthetic construct
MARSNLPLALGLALVAFCLLALPRDARARPQERMVGELRDLSPDDPQVQKAAQAAVASYNMGSNSIYYFRDTHIIKAQSQLVAGIKYFLTMEMGSTDCRKTRVTGDHVDLTTCPLAAGAQQEKLRCDFEVLVVPWQNSSQLLKHNCVQM

SEQ ID NO: 2 - DNA, 396 bp, synthetic construct
  source 1..396: mol_type = other DNA
atggcgcgttcgaacctcccgctggcgctgggcctggccctggtcgcattctgcctcctggcgctgccacgcgatgcccgggcccggccgcaggagcgcatggtcggagaactccgggacctgtcgcccgacgacccgcaggtgcagaaggcggcgcaggcggccgtggccagctacaacatgggcagcaacagcatctactacttccgagacacgcacatcatcaaggcgcagagccagctggtggccggcatcaagtacttcctgacgatggagatggggagcacagactgccgcaagaccagggtcactggagaccacgtcgacctcaccacttgccccctggcagcaggggcgcagcaggagctgcgctgtgactttgaggtccttgtggtt

SEQ ID NO: 3 - protein, 148 aa, synthetic construct
MARSNLPLALGLALVAFCLLALPRDARARPQERMVGELRDLSPDDPQVQKAAQAAVASYMGSNSIYYFRDTHIIKAQSQLVAGIKYFLTMEMGSTDCRKTRVTGDHVDLTTCPLAAGAQQEKLRCDFEVLVVPWQNSSQLLKHNCVQM

SEQ ID NO: 4 - DNA, 592 bp, synthetic construct
  source 1..592: mol_type = other DNA
actcacggctctgagggctccgacggcactgacggccatggcgcgttcgaacctcccgctggcgctgggcctggccctggtcgcattctgcctcctggcgctgccacgcgacgcccgggcccggccgcaggagcgcatggtcggagaactccgggacctgtcgcccgacgacccgcaggtgcagaaggcggcgcaggcggccgtggccagctacaacatgggcagcaacagcatctactacttccgagacacgcacatcatcaaggcgcagagccagctggtggccggcatcaagtacttcctgacgatggagatggggagcacagactgccgcaagaccagggtcactggagaccacgtcgacctcaccacttgccccctggcagcaggggcgcagcaggagaagctgcgctgtgactttgaggtccttgtggttccctggcagaactcctctcagctcctaaagcacaactgtgtgcagatgtgataagtccccgagggcgaaggccattgggtttggggccatggtggagggcacttcaggtccgtgggccgtatctgtcacaataaatggccagtgctgcttcttgca

SEQ ID NO: 5 - DNA, 2453 bp, synthetic construct
  source 1..2453: mol_type = other DNA
gcggccgcaagctcggcactcacggctctgagggctccgacggcactgacggccatggcgcgttcgaacctcccgctggcgctgggcctggccctggtcgcattctgcctcctggcgctgccacgcgatgcccgggcccggccgcaggagcgcatggtcggagaactccgggacctgtcgcccgacgacccgcaggtgcagaaggcggcgcaggcggccgtggccagctacaacatgggcagcaacagcatctactacttccgagacacgcacatcatcaaggcgcagagccaggtgcggcgggcggggtgctgggaggggacacccggcccagatgggggaggccacaggcgctgccccagcgtgcatgaagggggcctaaaagcgcaatcgggatattttcatgcaatattttaaaaatcgaattaatgctaaaactccacgatggacaaccatctaaatttcaaggaaagacataattaaaccctgtattgcactacctgccttcctggcttgcctggtcctagtctctgccctgatggggtggatcggggaggagggaaggcaggtggggacagtgggcagctccctagatggggcatgtctgatgtctgccaaggctgctggtggggctcagcctccaggcctctccaccctccatccccaccagaactcccccccacaccccccccacccccccccccaccccgtctgaatcatcccctctccctctctccccacctgagtgcctgggggcaacaggagaatatatcaagaggtgagaagtgaggatcaagggtcaagacccctgacctgcccctaccctatgcccccagctggtggccggcatcaagtacttcctgacgatggagatggggagcacagactgccgcaagaccagggtcactggagaccacgtcgacctcaccacttgccccctggcagcaggggcgcagcaggaggtaacagctgggctcctccagccccagccctccccagagcctcaggcactcaggttgtccatcctgaactggtttggctggacacgtagatgtctagatgtctggctgaacctgtcgtccttctggatgagtcagcctctgggccaagatggggtgcagaaaggaagctggggcttccctcgggaatggggaagttggctaccaagatctggagtctagccccagacatgcggcttgacatccgttgggtcagtgattgtccctctcttggcctggagcggcctggcaggcagagggctggctgttgggaggagacaggtcgaggctgggctcacccctccttctcccccatattccctccacagaagctgcgctgtgactttgaggtccttgtggttccctggcagaactcctctcagctcctaaagcacaactgtgtgcagatgtgataagtccccgagggcgaaggccattgggtttggggccatggtggagggcacttcaggtccgtgggccgtatctgtcacaataaatggccagtgctgcttcttgcattggtttcttccaagtgcttcgcttctgcccccatcccccactcccacttctggcgagctcccagctccccagcccaagctgagaggtcaccctgccacctgcagcagagctgctgctgtcccagcccagacagtgctgaaggtgcagggctgagcctccaaatgtggagcccccaaacaccccagcatcacaggcatagaggaacaaaccttagagcccccacacacagccagagactcccaatattctcagaggtcaggggtgggggcagactcacagaggaggggcgatggcctgagataagaacagggccggctgcctgggagcactctgaggaggagtctgtgctgtgctcccactcagccccaggaggagctcatgggtggcatggggggcttcaatcaggcagtgtcctcacccaggacactgagctcccagcctataaagattcctccacctgcatgtggcccaacgatggcagagtccctgcaggccaagagaccatgaaggctaggacagtgcctctcagtccacactgtgggctccacgccacaaggacgcctctcttgccttgccctgggcaagacgcctgaaaggggctagactaggagccgcagagggacagagattgtctagcgcccacaggaggggtgaggggacagattcaatgtaatgtcatttaaagaaaacagcattgcagccggcctcacacctgtaatcccagcactttggtaggctgaggtggatggatcacctgaggtcagggggttcaagaccagcctggtggccgggcgcggtggctcacgcctgtaatcccagcactttgggaggccgaggcgggcagatcacgaggtcagcagatcgagaccatcctgggtaacacggtgaaaccccgtctctactaaaaatacaaaaaattagctgggtgtagtggcg

(19) United States
(12) Patent Application Publication
(10) Pub. No.: US 2025/0073322 A1
(43) Pub. Date: Mar. 6, 2025

(54) CYSTATIN RNA COMPOSITIONS FOR TISSUE ENGINEERING

(71) Applicant: University of lowa Research Foundation, Iowa City, IA (US)

(72) Inventors: Aliasger K. Salem, Coralville, IA (US); Pornpoj Phruttiwanichakun, lowa City, IA (US)

(21) Appl. No.: 18/724,851

(22) PCT Filed: Dec. 29, 2022
(86) PCT No.: PCT/US2022/082553

§ 371 Date: Jun. 27, 2024

Related U.S. Application Data
(60) Provisional application No. 63/294,465, filed on Dec. 29, 2021.

(51) Int. Cl. A61K 38/57; A61K 48/00; A61P 19/08; C07K 14/81; C12N 15/88

(52) U.S. Cl. CPC A61K 38/57; A61K 48/0041; A61K 48/005; A61P 19/08; C07K 14/8139; C12N 15/88

(57) ABSTRACT

A composition comprising, for example, chemically modified RNA (cmRNA) encoding CST6 or a polypeptide with at least 80% amino acid sequence identity thereto, and methods of making and using the cmRNA, are provided.

### CROSS-REFERENCE TO RELATED APPLICATIONS

This application claims the benefit of the filing date of U.S. application No. 63/294,465, filed on Dec. 29, 2021, the disclosure of which is incorporated by reference herein.

### INCORPORATION BY REFERENCE OF SEQUENCE LISTING

A Sequence Listing is provided herewith as an xml file, “2297418.xml” created on Dec. 28, 2022 and having a size of 9,171 bytes. The content of the xml file is incorporated by reference herein in its entirety.

### BACKGROUND

On an annual basis, in the United States, bone grafts are the second most transplanted tissues/material, next to blood (Boyce et al., 1999). It is estimated that more than half a million bone grafting procedures are performed annually in the United States alone and close to 2.2 million world-wide (Giannoudis et al., 2005). Bone grafts and their substitutes have become an integral part of orthopedics and dentistry and are used in a wide range of clinical situations (Moussa & Dym, 2020). Though several strategies such as the use of morphogenetic proteins and growth factors have been introduced into the clinical practice, the alarming rise in its off-label use (Woo, 2012) and the rising complications following their use both in approved and off-label indications (Woo, 2012), points to the existing need for safer and more targeted approaches. In addition to carcinogenesis concerns, there are also documented adverse effects from using recombinant human bone morphogenetic protein-2 (rhBMP-2) in orthopedics including: vertebral osteolysis, ectopic/heterotopic bone and hematoma formation, dysphagia and neck swelling (Tannoury, 2014). Apart from safety concerns, the required supra-physiological dosage of rhBMP-2 needed to compensate for short half-lives also increases the overall treatment cost. Therefore, there exists a demand in both medicine and dentistry to develop regenerative strategies and biomaterials that are safe, predictable and cost-effective that could potentially overcome the barriers associated with other therapies (Laird et al., 2021).

Gene therapy is one approach to mitigate the barriers encountered with protein use but has its own set of challenges, including, low transfection efficiencies and safety concerns associated with non-viral and viral DNA approaches, respectively, although gene therapy studies conducted in animals using viral vectors delivered through a traditional ex vivo or an in vivo approach have successfully demonstrated that delivery of single or multiple transgenes (e.g., BMP-2 and BMP-7) is feasible and effective for bone regeneration applications (Evans et al., 2012; Evans, 2010). In spite of its proven efficacy in animal studies, it is well known that conducting human clinical trials and ultimately translating gene therapy into clinical settings especially for non-lethal conditions can be extremely challenging (Evans et al., 2012; Evans, 2010).

Another approach for improved therapeutics promoting fracture healing and bone regeneration has led to the introduction and rapid expansion of biomimetic materials (Deschaseaux et al., 2010; Jha et al., 2015; Wang et al., 2015; Kim et al., 2015; Vo et al., 2015; Quinlan et al., 2015; Suliman et al., 2015; Do et al., 2015) in conjunction with growth factors or morphogens, such as bone morphogenetic protein-2 (BMP-2) (Canalis et al., 1988; Seo et al., 2015; Quinlan et al., 2015; Karfeld-Sulzer at al., 2015; Atluir et al., 2015). BMP-2 delivered as a human recombinant protein on an absorbable collagen sponge (INFUSE® Bone Graft, Medtronic Spinal and Biologics, Memphis, TN) was shown to be effective in the treatment of patients with degenerative disc disease, bone fractures, as well as oral and maxillofacial osseous defects (Boyne et al., 2005: Khan et al., 2004). However, there are a number of drawbacks to using recombinant BMP-2 for both approved and off-label indications (Cancedda et al., 2007; Woo et al., 2012) as discussed above.

Therefore, there is a demand to develop regenerative strategies that are safe, cost-effective and that could potentially overcome the barriers associated with current approaches.

### SUMMARY

Chemically modified RNAs (cmRNAs) are short transcript sequences encoding a protein of interest and these transcripts, after entering cells, without requiring nuclear entry, undergo translation in the cytoplasm, thereby resulting ion synthesis of the protein of interest. Certain protein-based protease inhibitors may have the potential for promoting bone regeneration. Delivery of cmRNA encoding a cystatin, e.g., CST6 (cmRNA (CST6)), or structurally related molecules, is a strategy in regenerative medicine. As described herein, in one embodiment, cmRNA CST6 is encapsulated in stable nucleic acid lipid particles (SNALP), and SNALP-cmRNA (CST6) is delivered into bone defects, e.g., using a scaffold such as collagen matrix (CM). In vivo delivery of cmRNA (CST6) into osseous defects promotes significant bone regeneration.

The disclosure thus provides a therapeutic and a delivery system that may overcome barriers that exist with both protein-based, as well as-DNA based, therapeutics. Employing biomaterials to release chemically modified ribonucleic acid (cmRNA) in a controlled manner addresses the high cost and safety concerns existing with some recombinant protein-based approaches. By eliminating the need for nuclear trafficking (the ultimate barrier for successful DNA transfection in non-dividing cells), cmRNA delivery addresses the lower transfection efficiency associated with viral or other non-viral gene delivery systems. In one embodiment, the present strategy employs non-viral delivery vehicles, which alleviates the immunogenic concern that exists with viral vectors. Moreover, the in vivo approach rather than ex vivo transfection of cells to be modified may further reduce the overall cost.

In one embodiment, the system provides for chemically modified RNA molecules encoding a cystatin or a structurally related protein, for instance, cystatin M (CST6), which can be delivered to an area of therapeutic interest in the body, to promote bone regeneration, thereby improving healing. Exemplary polypeptides encoded by the cmRNA include but are not limited to

(SEQ ID NO: 1)
MARSNLPLALGLALVAFCLLALPRDARARPQERMVGELRDLS
PDDPQVQKAAQAAVASYNMGSNSIYYFRDTHIIKAQSQLVAG
IKYFLTMEMGSTDCRKTRVTGDHVDLTTCPLAAGAQQEKLRC
DFEVLVVPWQNSSQLLKHNCVQM
or
(SEQ ID NO: 3)
MARSNLPLAL GLALVAFCLL ALPRDARARP QERMVGELRD
LSPDDPQVQK AAQAAVASY MGSNSIYYFR DTHIIKAQSQ
LVAGIKYFLT MEMGSTDCRK TRVTGDHVDL TTCPLAAGAQ
QEKLRCDFEV LVVPWQNSSQ LLKHNCVQM,
or a

polypeptide having at least 80%, 85%, 88%, 90%, 92%, 94%, 95%, 97%, or 99% amino acid sequence identity thereto, including a polypeptide having 1, 2, 3, 4, 5, 6, or 7 substitutions relative to SEQ ID NO: 1 or 3.

An exemplary nucleotide sequence for a cmRNA includes but is not limited to:

(SEQ ID NO: 2)
Atggcg cgttcgaacc tcccgctggc
gctgggcctg gccctggtcg cattctgcct
cctggcgctgccacgcgatg cccgggcccg
gccgcaggag cgcatggtcg gagaactccg
ggacctgtcgcccgacgacc cgcaggtgca
gaaggcggcg caggcggccg tggccagcta
caacatgggcagcaacagca tctactactt
ccgagacacg cacatcatca aggcgcagag
ccagctggtggccggcat caagtacttc
ctgacgatgg agatggggag cacagactgc
cgcaagaccagggtcactgg agaccacgtc
gacctcacca cttgccccct ggcagcaggg
gcgcagcaggag ctgcgctgtg actttgaggt
ccttgtggtt
or
(SEQ ID NO: 4)
actcacggct ctgagggctc cgacggcact
gacggccatg gcgcgttcga acctcccgct
ggcgctgggc ctggccctgg tcgcattctg
cctcctggcg ctgccacgcg acgcccgggc
ccggccgcag gagcgcatgg tcggagaact
ccgggacctg tcgcccgacg acccgcaggt
gcagaaggcg gcgcaggcgg ccgtggccag
ctacaacatg ggcagcaaca gcatctactc
cttccgagac acgcacatca tcaaggcgca
gagccagctg gtggccggca tcaagtactt
cctgacgatg gagatgggga gcacagactg
ccgcaagacc agggtcactg gagaccacgt
cgacctcacc acttgccccc tggcagcagg
ggcgcagcag gagaagctgc gctgtgactt
tgaggtcctt gtggttccct ggcagaactc
ctctcagctc ctaaagcaca actgtgtgca
gatgtgataa gtccccgagg gcgaaggcca
ttgggtttgg ggccatggtg gagggcactt
caggtccgtg ggccgtatct gtcacaataa
atggccagtg ctgcttcttg ca

where “t” is “u”, or a nucleotide sequence with at least 80%, 85%, 88%, 90%, 92%, 94%, 95%, 97%, or 99% nucleic acid sequence identity thereto, that encodes a polypeptide having t least 80% amino acid sequence identity to SEQ ID NO: 1 or 3, wherein the modification(s) include nucleotides with modified bases, modified sugars and/or non-phosphodiester bonds.

The system includes a composition having chemically modified RNA molecules, e.g., cmRNA, a non-viral delivery vehicle suitable for encapsulating or complexing with the modified RNA, e.g., cmRNA complexed with lipids or a synthetic polymer such as a dendrimer, and optionally a scaffold, e.g., collagen for the delivery vehicle. In one embodiment, the scaffold is biocompatible. In one embodiment, the scaffold is biocompatible and bioresorbable (biodegradable). The scaffold allows for sustained and targeted in vivo delivery of the cmRNA to the physiological site of interest. In one embodiment, the scaffold provides anchorage that maintains the complexed or encapsulated cmRNA molecules for a period of time in desired tissue and the delivery vehicle for the cmRNA molecule, which are complexed with or encapsulated in the delivery vehicle, releases, e.g., over time, the cmRNA to the desired tissue. Although in one embodiment a non-viral vector may be employed to deliver RNA, viral vectors may also be employed. In one embodiment, the cmRNA is cmRNA (CST6) that is complexed to or encapsulated in lipids or a synthetic polymer, which cmRNA is released from an implanted scaffold and is taken up by local cells that in turn express the encoded product (CST6 protein). Any combination of viral and non-viral delivery vehicles, and scaffolds, e.g., made from natural or synthetic polymers, may be used to deliver cmRNA.

As disclosed herein, in one embodiment, a cmRNA encoding CST6 was synthesized and mixed with molecules for delivery (delivery vehicle) of the cmRNA. In one embodiment, a composition is provided comprising cmRNA, a non-viral delivery vehicle and a scaffold. In one embodiment, the cmRNA encodes CST6 or a protein with at least 80%, 85%, 87%, 90%, 92%, 95%, 98% or 99% amino acid sequence identity thereto. In one embodiment, the cmRNA in the composition is present in an amount that enhances bone regeneration. In one embodiment, the delivery vehicle comprises a plurality of different lipids. In one embodiment, the delivery vehicle comprises a synthetic polymer, e.g., comprising PEI, poly(lactic-co-glycolic acid) (PLGA) or polyamidoamine (PAMAM). In one embodiment, the delivery vehicle comprises a natural polymer, e.g., chitosan or cyclodextrin. In one embodiment, the delivery vehicle comprises a cationic polymer, for instance, PEI, chitosan, cyclodextrin or dendrimers. In one embodiment, the delivery vehicle comprises a non-cationic polymer, e.g., dioleoylphosphatidyl ethanolamine (DOPE), cholesterol, PAMAM or poloxamer. In one embodiment, the cmRNA is complexed with a cationic polymer and encapsulated into microparticles, e.g., PLGA microparticles. In one embodiment, the cmRNA is embedded in the delivery vehicle. In one embodiment, the delivery vehicle comprises microparticles. In one embodiment, the cmRNA comprises 5-methylcytidine-5′-triphosphate. In one embodiment, the cmRNA comprises pseudoundine-5′-triphosphate. In one embodiment, the scaffold comprises a synthetic polymer or a natural polymer. In one embodiment, the scaffold is biocompatible and bioresorbable. In one embodiment, the scaffold comprises collagen.

In one embodiment, the disclosure provides for a composition comprising isolated DNA encoding CST6. In one embodiment, the DNA encodes CST6 comprising SEQ ID NO: 1 or 3, or a fragment thereof with the same activity, or a protein with at least 80%, 85%, 87%, 90%, 92%, 95%, 98% or 99% amino acid sequence identity thereto. In one embodiment, the DNA in the composition is present in an amount that enhances bone regeneration. In one embodiment, the DNA is a plasmid which is optionally in a delivery vehicle comprising a plurality of different lipids. In one embodiment, the delivery vehicle comprises a synthetic polymer, e.g., comprising PEI, poly(lactic-co-glycolic acid) (PLGA) or polyamidoamine (PAMAM). In one embodiment, the delivery vehicle comprises a natural polymer, e.g., chitosan or cyclodextrin. In one embodiment, the delivery vehicle comprises a cationic polymer, for instance, PEI, chitosan, cyclodextrin or dendrimers. In one embodiment, the delivery vehicle comprises a non-cationic polymer, e.g., dioleoylphosphatidyl ethanolamine (DOPE), cholesterol, PAMAM or poloxamer. In one embodiment, the cmRNA is complexed with a cationic polymer and encapsulated into microparticles, e.g., PLGA microparticles. In one embodiment, the DNA is embedded in the delivery vehicle. In one embodiment, the delivery vehicle comprises microparticles. In one embodiment, the DNA is a viral DNA vector, e.g., an AAV or lentivirus vector. In one embodiment, the DNA and delivery vehicle are encapsulated or complexed with a scaffold comprising a synthetic polymer or a natural polymer. In one embodiment, the scaffold is biocompatible and bioresorbable. In one embodiment, the scaffold comprises collagen.

Also provided are methods of making the compositions, e.g., by contacting, for instance, mixing, nucleic said such as the modified RNA molecules, and a delivery vehicle, to form complexes or particles, which in turn are optionally introduced to a scaffold.

Further provided is a method to enhance tissue regeneration, e.g., bone regeneration, in vivo or ex vivo. The method includes introducing the composition to a site in a mammal in need of repair or augmentation. In one embodiment, the tissue is a bone. In one embodiment, the composition is placed at a site of a bone defect (an osseous defect). In one embodiment, bone density at the defect site is increased. In one embodiment, the defect is in a jawbone, ankle bone, ulna, radius, humerus, skull, femur or tibia, or any other bone or skeletal defect. In one embodiment, the administration of the composition increases bone regeneration. In one embodiment, the mammal is in need of spinal fusion, fracture healing, delayed union, non-union, periodontal regeneration, ridge preservation, alveolar ridge augmentation, peri-implant bone regeneration or sinus augmentation.

### BRIEF DESCRIPTION OF THE FIGURES

FIG. 1. Schematic showing general mechanism of cmRNA based production of regenerative proteins.

FIG. 2. CST6 upregulates osteogenic gene expression. BMSCs were incubated with rCST6 protein at 10 ng and 50 ng. RT-PCR analysis showed the relative levels of expression of ALP, RUNX2 and OS.

FIG. 3. An example of a rat traversing the walkway and the corresponding pawprints recorded using CatWalkXT.

FIG. 4. Manual measurement of print length (PL), toe spread (TS), and intermediate toe spread (ITS).

FIG. 5. Post-op radiograph of plated 6 mm defect in the femur.

FIG. 6. Coronal and sagittal slices of a healing defect showing new trabecular bone architecture filing the 6 mm gap.

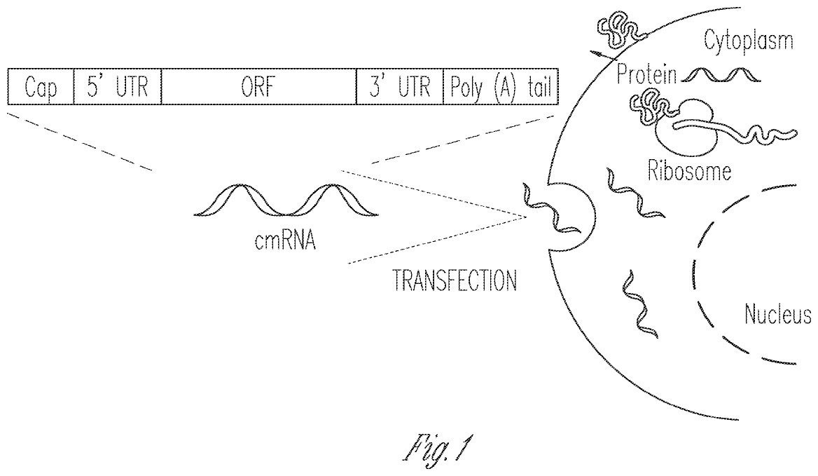

### DETAILED DESCRIPTION

Identification and characterization of molecules involved in bone development and fracture healing has led to the introduction and rapid expansion of biomimetic materials. CST6 was negatively correlated with bone lytic lesions in multiple myeloma. Another study reported a negative correlation between the expression and secretion of CST6 and metastatic bone lytic lesions in breast cancer (Jin et al., 2012). Thus, CST6 is a molecule that may promote bone regeneration.

CST6 is a protein from the protease inhibitor based cystatin family. CST6 expression is restricted to cutaneous epithelia and is secreted as a 14-Kda and a 17-Kda glycosylated protein. Recombinant CST6 protein inhibited osteoclast maturation. The use of chemically modified RNA (cmRNA) encoding proteins is a strategy with potential to overcome the barriers associated with protein and gene therapy. Thus, delivery of cmRNA encoding CST6 (cmRNA (CST6)) is an attractive strategy in regenerative medicine. For example, cmRNA (CST6) may be encapsulated in stable nucleic acid lipid particles (SNALP) that are delivered into bone defects from a collagen matrix (CM), which in turn promotes bone regeneration, e.g., at a site of an osseous defect.

For example, as described hereinbelow, to test whether SNALP-cmRNA (CST6) nanoparticles (NPs) inhibit osteoclast maturation, plasmid DNA (pDNA) encoding CST6 is used as a template to synthesize cmRNA (CST6). Following this, the lack of immunogenicity of cmRNA (CST6) is verified and then loaded into SNALPs. The in vitro cytotoxicity and transfection efficiency of SNALP-cmRNA (CST6) NPs is evaluated in RAW264.7 and bone marrow-derived macrophages (BMM) to characterize osteoclast maturation inhibition activity. The osteoblastic differentiation of treated cells (with appropriate controls) is determined by evaluating the expression of bone specific genes (collagen type I, RUNX2, VEGF, alkaline phosphatase (ALP) and osteocalcin, core binding factor (Cbfa-1), CST6 and Osterix) at specific time points, post-treatment. Reduction of osteoclast maturation is monitored by evaluation of Tartrate-resistant acid phosphatase (TRAP) staining and RT-PCR analysis for NTATc1, TRAP, MMP-9, OSCAR, DC-STAMP, ATP6vOd2 and Cathepsin K. To quantify protein production, the amount of CST6 produced by the cells is determined by ELISA. Functionality of transfection is examined by assessing bone nodule formation using Alizarin red S staining and by determining alkaline phosphatase (ALP) activity. In addition, the attachment and proliferation of BMSCs and MC3T3-E1 cells on CM with and without SNALP-cmRNA (CST6) NPs is assessed using imaging techniques.

In one embodiment, to determine if implantation of collagen matrix (CM) containing, for example, SNALP-cmRNA (CST6) NPs can induce bone formation, that delivery system is evaluated in, for example, unilateral diaphyseal femoral defects in Sprague Dawley rats to identify the effective dose (ED) of cmRNA (CST6) using the following groups: CM only, CM with SNALP, CM containing SNALP-cmRNA (CST6) in the following cmRNA doses: 25, 50, 75 and 100 μg. Following that, bone formation is assessed in the unilateral diaphyseal femoral defects in rats using the following groups: defects left empty, defects treated with: CM alone, CM containing SNALP, CM containing SNALP-cmRNA (CST6) with ED of cmRNA. Defects treated with CM containing: SNALP-scrambled cmRNA (CST6), recombinant protein form of CST6 (ED equivalent dose) with SNALP, PEI-plasmid DNA (ED equivalent dose) polyplexes encoding CST6 and CM with BMP-2 protein serve as controls (n=16 per group). Eight rats from each group will undergo analysis for gene expression using either RT-PCR (n=5) and histology (n=3) evaluating markers for angiogenesis, osteoclast inhibition and osteogenic differentiation in addition to micro-CT analysis at week 12. Eight rats from each implant group at 12 weeks are subjected to mechanical testing (torsion to failure) to determine strength and stiffness of the healed bone. All 16 rats will undergo gait analysis.

### Nucleic Acid Molecules for Use in the Compositions

The disclosure provides for isolated nucleic acid, e.g., DNA or RNA such as a plasmid or viral vector and modified RNA molecules comprising, for example, one or more modified nucleosides or modified nucleotides, such as 2-thiouridine and/or 5-methylcytidine, or complexes or particles comprising the DNA or modified RNA molecules, methods of using the isolated nucleic acid, complexes or particles, and methods of using scaffolds having the isolated nucleic acid, complexes or particles. In one embodiment the modified nucleotide is 2-thiouridine. In one embodiment, is 5-methylcytidine. In one embodiment the modified nucleotide is pseudouridine (Ψ), which includes m¹acp³Ψ (1-methyl-3-(3-amino-5-carboxypropyl) pseudouridine, m¹Ψ (1-methylpseudouridine), Ψm (2′-O-methylpseudouridine, m⁵D (5-methyldihydrouridine) or m³Ψ (3-methylpseudouridine). In one embodiment, the term modified nucleotide refers to a monophosphate, diphosphate, or triphosphate of any modified base or sugar of a nucleoside, or a nucleotide having non-phosphodiester bonds.

In one embodiment, the present disclosure provides a cmRNA comprising at least one 2-thiouridine and/or 5-methylcytidine residue. In one embodiment, the cmRNA encodes a protein of interest. In one embodiment, the present disclosure provides in vitro transcribed RNA molecules comprising a plurality of 2-thiouridine and/or 5-methylcytidine. In one embodiment, the present invention provides a cmRNA molecule encoding CST6, where the residues in the cmRNA molecule comprises 5% to 15%, e.g., about 10%, 2-thiouridine and/or 5-methylcytidine residue.

In one embodiment, the present disclosure provides an in vitro synthesized RNA polynucleotide comprising 2-thiouridine and/or 5-methylcytidine, or comprising one or more of m⁵C, m⁵U, m⁶A, s²U, or 2′-O-methyl-U. In another embodiment, the present disclosure provides an in vitro synthesized RNA polyribonucleotide comprising m⁵U, m⁶A, s²U, Ψ, or 2′-O-methyl-U, or any combination thereof.

In one embodiment, the RNA molecule further comprises a poly-A tail. In another embodiment, the RNA molecule does not comprise a poly-A tail.

In one embodiment, the RNA molecule further comprises a m7 GpppG cap. In another embodiment, the RNA molecule does not comprise a m7 GpppG cap.

In one embodiment, the RNA molecule further comprises a cap-independent translational enhancer. In another embodiment, the RNA molecule does not comprise a cap-independent translational enhancer. In another embodiment, the cap-independent translational enhancer is a tobacco etch virus (TEV) cap-independent translational enhancer. In one embodiment, the cap-independent translational enhancer is any other cap-independent translational enhancer known in the art.

In one embodiment, the nucleoside that is modified in a RNA molecule is uridine (U). In one embodiment, the modified nucleoside is cytidine (C). In one embodiment, the modified nucleoside is adenine (A). In another embodiment the modified nucleoside is guanine (G). In one embodiment, the modified nucleoside is m⁵C (5-methylcytidine). In one embodiment, the modified nucleoside is m⁵U (5-methyluridine). In one embodiment, the modified nucleoside is m⁶A (N⁶-methyladenosine). In one embodiment, the modified nucleoside is s²U (2-thiouridine). In one embodiment, the modified nucleoside is Ψ (pseudouridine). In one embodiment, the modified nucleoside is Um (2′-O-methyluridine). In one embodiment, the RNA molecule may include combinations of modified nucleosides.

In one embodiment, the modified nucleoside is m⁵C (5-methylcytidine). In one embodiment, the modified nucleoside is m⁵U (5-methyluridine). In one embodiment, the modified nucleoside is m⁶A (N⁶-methyladenosine). In one embodiment, the modified nucleoside is s²U (2-thiouridine). In one embodiment, the modified nucleoside is Ψ (pseudouridine). In one embodiment, the RNA molecule may include combinations of modified nucleosides.

In certain embodiments, up to approximately 100% of the residues in the RNA molecule are modified, for instance, up to approximately 70% of the residues modified. In another embodiment, approximately up to 65% of the residues are modified. In another embodiment, approximately up to 60% of the residues are modified. In another embodiment up to approximately 55% of the residues are modified. In another embodiment, approximately up to 50% of the residues are modified. In another embodiment, approximately up to 45% of the residues are modified. In another embodiment, approximately up to 40% of the residues are modified. In another embodiment, approximately up to 35% of the residues are modified. In another embodiment, approximately up to 30% of the residues are modified. In another embodiment, approximately up to 25% of the residues are modified. In another embodiment, approximately up to 20% of the residues are modified. In another embodiment, approximately up to 15% of the residues are modified. In another embodiment, approximately up to 10% of the residues are modified. In another embodiment, approximately up to 5% of the residues are modified. In another embodiment, approximately up to 2.5% of the residues are modified. In another embodiment, approximately up to 1% of the residues are modified.

A RNA molecule according to the disclosure with increased stability and diminished immunogenicity may be produced with a nucleotide mixture which contains both unmodified and also modified nucleotides, where 5 to 50% of the cytidine nucleotides and 5 to 50% of the uridine nucleotides are modified. The adenosine- and guanosine-containing nucleotides can be unmodified. A nucleotide mixture can also be used wherein some of the ATPs and/or GTPs are also modified, where their content should not exceed 20% and where their content, if present, may lie in a range from 0.5 to 10%. In one embodiment, a mRNA is provided which has 5 to 50% of modified cytidine nucleotides and 5 to 50% of uridine nucleotides and 50 to 95% of unmodified cytidine nucleotides and 50 to 95% of unmodified uridine nucleotides, and the adenosine and guanosine nucleotides can be unmodified or partially modified, and they may be present in unmodified form.

In one embodiment, 10 to 35% of the cytidine and uridine nucleotides are modified, for instance, the content of the modified cytidine nucleotides lies in a range from 7.5 to 25% and the content of the modified uridine nucleotides in a range from 7.5 to 25%. A relatively low content, e.g., only 10% each or a total, of modified cytidine and/or uridine nucleotides may achieve the desired properties.

The nature of the modification of the nucleosides has an effect on the stability and hence the lifetime and biological activity of the mRNA. Suitable modifications are set out in Table 1:

TABLE 1

| | Base | Sugar | Naturally |
|---|---|---|---|
| | modification | modification | in |
| Name | (5-position) | (2′-position) | mRNA |
| Uridine | | | |
| 5-methyluridine 5′-triphosphate | CH3 | — | no |
| (m5U) | | | |
| 5-idouridine 5′-triphosphate (15U) | I | — | no |
| 5-bromouridine 5′-triphosphate | Br | | no |
| (Br5U) | | | |
| 2-thiouridine 5′-triphosphate (S4U) | S (in 2 | — | no |
| | position) | | |
| 4-thiouridine 5′-triphosphate (S2U) | S (in 4 | — | no |
| | position) | | |
| 2′-methyl-2′-deoxyuridine 5′- | — | CH₃ | yes |
| triphosphate (U2′m) | | | |
| 2′-amino-2′-deoxyuridine 5′- | — | NH₂ | no |
| triphosphate (U2′NH2) | | | |
| 2′-azido-2′-deoxyuridine 5′- | — | N₃ | no |
| triphosphate (U2′N3) | | | |
| 2′-fluoro-2′-deoxyuridine 5′- | — | F | no |
| triphosphate (U2′F) | | | |
| Cytidine | | | |
| 5-methylcytidine 5′-triphosphate | CH₃ | — | yes |
| (m5C) | | | |
| 5-idocytidine 5′-triphosphate (15U) | I | — | no |
| 5-bromocytidine 5′-triphosphate | Br | — | no |
| (Br5U) | | | |
| 2-thiocytidine 5′-triphosphate (S2C) | S (in 2 | — | no |
| | position) | | |
| 2′-methyl-2′-deoxycytidine 5′- | — | CH₃ | yes |
| triphosphate (C2′m) | | | |
| 2′-amino-2′-deoxycytidine 5′- | — | NH₂ | no |
| triphosphate (C2′NH2) | | | |
| 2′-azido-2′-deoxycytidine 5′- | — | N₃ | no |
| triphosphate (C2′N3) | | | |
| 2′-fluoro-2′-deoxycytidine 5′- | — | F | no |
| triphosphate (C2′F) | | | |
| Adenosine | | | |
| N6-methyladenosine 5′-triphosphate | CH₃ (in 6 | — | yes |
| (m6A) | position) | | |
| N1-methyladenosine 5′-triphosphate | CH₃ (in 1 | — | no |
| (m1A) | position) | | |
| 2′-O-methyladenosine 5′- | — | CH₃ | yes |
| triphosphate (A2′m) | | | |
| 2′-amino-2′-deoxyadenosine 5′- | — | NH₂ | no |
| triphosphate (A2′NH2) | | | |
| 2′-azido-2′-deoxyadenosine 5′- | — | N₃ | no |
| triphosphate (A2′N3) | | | |
| 2′-fluoro-2′-deoxyadenosine 5′- | — | F | no |
| triphosphate (A2′F) | | | |
| Guanosine | | | |
| N1-methylguanosine 5′-triphosphate | CH₃ (in 1 | — | no |
| (m1G) | position) | | |
| 2′-O-methylguanosine 5′- | — | CH₃ | yes |
| triphosphate (G2′m) | | | |
| 2′-amino-3′-deoxyguanosine 5′- | — | NH₂ | no |
| triphosphate (G2′NH2) | | | |
| 2′-azido-2′-deoxyguanosine 5′- | — | N₃ | no |
| triphosphate (G2′N3) | | | |
| 2′-fluoro-2′-deoxyguanosine 5′- | — | F | no |
| triphosphate (G2′F) | | | |

In one embodiment, either all uridine nucleotides and cytidine nucleotides can each be modified in the same form or else a mixture of modified nucleotides can be used for each. The modified nucleotides can have naturally or not naturally occurring modifications. A mixture of various modified nucleotides can be used. Thus, for example one part of the modified nucleotides can have natural modifications, while another part has modifications not occurring naturally or a mixture of naturally occurring modified and/or not naturally occurring modified nucleotides can be used. Also, a part of the modified nucleotides can have a base modification and another part a sugar modification. In the same way, it is possible that all modifications are base modifications or all modifications are sugar modifications or any suitable mixture thereof. By variation of the modifications, the stability and/or duration of action of the RNA can be selectively adjusted.

In one embodiment, at least two different modifications are used for one type of nucleotide, where one type of the modified nucleotides has a functional group via which further groups can be attached. Nucleotides with different functional groups can also be used, in order to provide binding sites for the attachment of different groups. Thus, for example a part of the modified nucleotides can bear an azido group, an amino group, a hydroxy group, a thiol group or some other reactive group which is suitable for reaction under predefined conditions. The functional group can also be such that it can under certain conditions activate a naturally present group capable of binding, so that molecules with functions can be coupled. Nucleotides which are modified so that they provide binding sites can also be introduced as adenosine or guanosine modifications. The selection of the particular suitable modifications and the selection of the binding sites to be made available depends on what groups are to be introduced and with what frequency these are to be present. Thus, the content of the nucleotides provided with functional and/or activating groups depends on how high the content of groups to be coupled is to be and can easily be determined by those skilled in the art. As a rule, the content of nucleotides modified with functional and/or activating groups, if present, is 1 to 25% of the modified nucleotides. Those skilled in the art can, if necessary, determine the most suitable groups in each case and the optimal content thereof by routine experiments.

In one embodiment, a combination of 2′-thiouridine as a modified uridine-containing nucleotide and/or 5′-methylcytidine as a modified cytidine nucleotide is/are employed. In one embodiment, these two nucleotides are each present at a content of about 10 to about 30%, or are present at a total content of about 7% to about 30%. Nucleotides modified in another way can optionally also be present, as long as the total content of modified nucleotides does not exceed 50% of the particular nucleotide type. In one embodiment, a polyribonucleotide has 5 to 50%, e.g., 5 to 30% or 7.5 to 25%, of the uridine nucleotides as 2′-thiouridine nucleotides, and 5 to 50%, e.g., 5 to 30% or 7.5 to 25%, of the cytidine nucleotides as 5′-methylcytidine nucleotides, where the adenosine and guanosine nucleotides can be unmodified or partially modified nucleotides. In one embodiment, this mRNA according to the invention additionally has a 7′-methylguanosine cap and/or a poly(A) end. Thus, in one embodiment the mRNA is produced in its mature form, e.g., with a GppG cap, an IRES and/or a polyA tail.

In many cases, as stated above, for the improvement of immunogenicity and stability or for adjustment of properties it can be beneficial to combine modified nucleosides with functional groups, which provide binding sites, with non-functionally modified nucleosides. If functionally modified nucleosides are desired, 2′-azido and 2′-amino nucleosides may be employed.

In one embodiment, the cytidine nucleotides and/or uridine nucleotides can have a modification which creates a binding site, such as for example azido, NH, SH or OH groups.

In one embodiment, the length of a RNA molecule is greater than 30 nucleotides in length. In another embodiment, the RNA molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides. In one embodiment, the length is less than 10,000 nucleotides. In one embodiment, the length is at least 100 nucleotides up to 8,000 nucleotides.

### Exemplary Proteins Encoded by the Isolated Nucleic Acid

Exemplary polypeptides encoded by the nucleic acid, e.g., cmRNA, include but are not limited to

(SEQ ID NO: 1)
MARSNLPLALGLALVAFCLLALPRDARARPQERMVGELR
DLSPDDPQVQKAAQAAVASYNMGSNSIYYFRDTHIIKAQ
SQLVAGIKYFLTMEMGSTDCRKTRVTGDHVDLTTCPLAA
GAQQEKLRCDFEVLVVPWQNSSQLLKHNCVQM
or
(SEQ ID NO: 3)
marsnlplal glalvafcll alprdararp qermvgelrd
lspddpqvqk aaqaavasy mgsnsiyyfr dthiikaqsq
lvagikyflt memgstderk trvtgdhvdl ttcplaagaq
qeklredfev lvvpwqnssq llkhncvqm,

or a polypeptide having at least 80%, 85%, 88%, 90%, 92%, 94%, 95%, 97%, or 99% amino acid sequence identity thereto, including a polypeptide having 1, 2, 3, 4, 5, 6, or 7 substitutions relative to SEQ ID NO: 1 or 3.

An exemplary nucleotide sequence for the polypeptide includes but is not limited to:

gcggccgcaa gctcggcact cacggctctg agggctccga
cggcactgac ggccatggcg cgttcgaacc tcccgctggc
gctgggcctg gccctggtcg cattctgcct cctggcgctg
ccacgcgatg cccgggcccg gccgcaggag cgcatggtcg
gagaactccg ggacctgtcg cccgacgacc cgcaggtgca
gaaggcggcg caggcggccg tggccagcta caacatgggc
agcaacagca tctactactt ccgagacacg cacatcatca
aggcgcagag ccaggtgcgg cgggcggggt gctgggaggg
gacacccggc ccagatgggg gaggccacag gcgctgcccc
agcgtgcatg aagggggcct aaaagcgcaa tcgggatatt
ttcatgcaat attttaaaaa tcgaattaat gctaaaactc
cacgatggac aaccatctaa atttcaagga aagacataat
taaaccctgt attgcactac ctgccttcct ggcttgcctg
gtcctagtct ctgccctgat ggggtggatc ggggaggagg
gaaggcaggt ggggacagtg ggcagctccc tagatggggc
atgtctgatg tctgccaagg ctgctggtgg ggctcagcct
ccaggcctct ccaccctcca tccccaccag aactcccccc
cacacccccc ccaccccccc ccccaccccg tctgaatcat
cccctctccc tctctcccca cctgagtgcc tgggggcaac
aggagaatat atcaagaggt gagaagtgag gatcaagggt
caagacccct gacctgcccc taccctatgc ccccagctgg
tggccggcat caagtacttc ctgacgatgg agatggggag
cacagactgc cgcaagacca gggtcactgg agaccacgtc
gacctcacca cttgccccct ggcagcaggg gcgcagcagg
aggtaacagc tgggctcctc cagccccagc cctccccaga
gcctcaggca ctcaggttgt ccatcctgaa ctggtttggc
tggacacgta gatgtctaga tgtctggctg aacctgtcgt
ccttctggat gagtcagcct ctgggccaag atggggtgca
gaaaggaagc tggggcttcc ctcgggaatg gggaagttgg
ctaccaagat ctggagtcta gccccagaca tgcggcttga
catccgttgg gtcagtgatt gtccctctct tggcctggag
cggcctggca ggcagagggc tggctgttgg gaggagacag
gtcgaggctg ggctcacccc tccttctccc ccatattccc
tccacagaag ctgcgctgtg actttgaggt ccttgtggtt
ccctggcaga actcctctca gctcctaaag cacaactgtg
tgcagatgtg ataagtcccc gagggcgaag gccattgggt
ttggggccat ggtggagggc acttcaggtc cgtgggccgt
atctgtcaca ataaatggcc agtgctgctt cttgcattgg
tttcttccaa gtgcttcgct tctgccccca tcccccactc
ccacttctgg cgagctccca gctccccagc ccaagctgag
aggtcaccct gccacctgca gcagagctgc tgctgtccca
gcccagacag tgctgaaggt gcagggctga gcctccaaat
gtggagcccc caaacacccc agcatcacag gcatagagga
acaaacctta gagcccccac acacagccag agactcccaa
tattctcaga ggtcaggggt gggggcagac tcacagagga
ggggcgatgg cctgagataa gaacagggcc ggctgcctgg
gagcactctg aggaggagtc tgtgctgtgc tcccactcag
ccccaggagg agctcatggg tggcatgggg ggcttcaatc
aggcagtgtc ctcacccagg acactgagct cccagcctat
aaagattcct ccacctgcat gtggcccaac gatggcagag
tccctgcagg ccaagagacc atgaaggcta ggacagtgcc
tctcagtcca cactgtgggc tccacgccac aaggacgcct
ctcttgcctt gccctgggca agacgcctga aaggggctag
actaggagcc gcagagggac agagattgtc tagcgcccac
aggaggggtg aggggacaga ttcaatgtaa tgtcatttaa
agaaaacagc attgcagccg gcctcacacc tgtaatccca
gcactttggt aggctgaggt ggatggatca cctgaggtca
gggggttcaa gaccagcctg gtggccgggc gcggtggctc
acgcctgtaa tcccagcact ttgggaggcc gaggcgggca
gatcacgagg tcagcagatc gagaccatcc tgggtaacac
ggtgaaaccc cgtctctact aaaaatacaa aaaattagct
gggtgtagtg gcg

(SEQ ID NO:2 represents the underlined nucleotides, e.g., those corresponding to mRNA, and SEQ ID NO:5 is the full length genomic sequence),

(SEQ ID NO: 4)
actcacggct ctgagggctc cgacggcact
gacggccatg gcgcgttcga acctcccgct
ggcgctgggc ctggccctgg tcgcattctg
cctcctggcg ctgccacgcg acgcccgggc
ccggccgcag gagcgcatgg tcggagaact
ccgggacctg tcgcccgacg acccgcaggt
gcagaaggcg gcgcaggcgg ccgtggccag
ctacaacatg ggcagcaaca gcatctacta
cttccgagac acgcacatca tcaaggcgca
gagccagctg gtggccggca tcaagtactt
cctgacgatg gagatgggga gcacagactg
ccgcaagacc agggtcactg gagaccacgt
cgacctcacc acttgccccc tggcagcagg
ggcgcagcag gagaagctgc gctgtgactt
tgaggtcctt gtggttccct ggcagaactc
ctctcagctc ctaaagcaca actgtgtgca
gatgtgataa gtccccgagg gcgaaggcca
ttgggtttgg ggccatggtg gagggcactt
caggtccgtg ggccgtatct gtcacaataa
atggccagtg ctgcttcttg ca

where “t” is “u” if the nucleic acid is RNA, and the modification(s) include nucleotides with modified bases, modified sugars and/or non-phosphodiester bonds.

Conservative amino acid substitutions may be employed—that is, for example, aspartic-glutamic as acidic amino acids; lysine/arginine/histidine as polar basic amino acids;
leucine/isoleucine/methionine/valine/alanine/proline/glycine non-polar or hydrophobic amino acids; serine/threonine as polar or hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

The disclosure also envisions a peptide or polypeptide with non-conservative substitutions. Non-conservative substitutions entail exchanging a member of one of the classes described above for another.

### Uses for the Nucleic Acid Molecules

According to the disclosure, a nucleic acid molecule, e.g., a RNA molecule where a portion of the nucleotides are modified nucleotides, and the use of that molecules for the production of a gene product for the treatment of diseases or disorders which can be moderated or cured by the provision of nucleic acid or proteins in vivo, are provided. In one embodiment, a RNA molecule with increased stability and/or decreased immunogenicity is provided for use in the systems of the invention. The RNA contains a ribonucleotide sequence which, in one embodiment, encodes a protein or fragment thereof whose function in the cell or in the vicinity of the cell is needed or beneficial, e.g., a protein the lack or defective form is a trigger for a disease or an illness, that can moderate or prevent a disease or an illness, or a can promote a process which is beneficial for the body, in a cell or its vicinity. In one embodiment, the RNA contains the sequence for the complete protein or a functional variant thereof. Further, the ribonucleotide sequence can encode a protein which acts as a factor, inducer, regulator, stimulator or enzyme, or a functional fragment thereof, where this protein is one whose function is necessary in order to remedy a disorder or in order to initiate processes in vivo such as the formation of new bone development, or other tissues, etc. Here, a functional variant can undertake the function of the protein whose function in the cell is needed or the lack or defective form thereof is pathogenic. In addition, the nucleic acid molecule can also have further functional regions and/or 3′ or 5′ noncoding regions. The 3′ and/or 5′ noncoding regions can be the regions naturally flanking the encoded protein or else artificial sequences which contribute to the stabilization of RNA. Those skilled in the art can discover the sequences suitable for this in each case by routine experiments.

In one embodiment, the RNA molecule may be used for the therapy of diseases or for the provision of proteins beneficial to the body. When the RNA molecule is used for the therapy of diseases, its expression in a cell in a tissue may leads to the moderation of an illness. For example, the RNA may encode a protein or protein fragment the presence thereof can moderate an illness or be beneficial or supportive to the body, for instance, because there is not sufficient protein or not sufficient function (nonpathogenic) protein or because the protein or fragment can benefit the body under certain conditions, e.g., in the treatment of defects or in the context of implantation. These include altered forms of proteins or protein fragments, i.e., forms of proteins which may alter in the course of the metabolism, e.g., matured forms of a protein, etc. Proteins which play a part in growth processes, which are for example necessary in controlled regeneration and can then be formed specifically by introduction of the mRNA according to the disclosure, can also be provided. This can, for example, be useful in growth processes or for the treatment of bone defects, tissue defects and in the context of implantation and transplantation.

Since biological substances very often have extremely short half-lives, it was previously necessary to use very high dosages of proteins, which burdens the patient with severe side effects. This disadvantage is avoided since using the RNA according to the disclosure the desired and/or needed proteins can be used selectively and suitably dosed. This decreases or even completely spares the patient the side effects. In this embodiment, the RNA which encodes desired and/or needed substances, can be applied onto the implant in a coating releasing the RNA in a measured manner and then released gradually therefrom in a measured manner, so that the cells in the vicinity of the implant can continuously or intermittently produce and, if necessary, release the desired factors. Polylactide or polylactide/glycolide polymers, PAMAM or lipids may, for example, be used as a delivery vehicle. In this way it is possible selectively to release the desired factors continuously, intermittently, over a longer or shorter time and at the desired site.

A further field in which the nucleic acid molecule according to the disclosure can be used is the field of regenerative medicine. Through disease processes or through aging, degenerative diseases arise which can be treated and moderated or even cured by introduction of proteins produced too little or not at all owing to the disease or aging processes. By introduction of a relevant nucleic acid encoding these proteins, the degenerative process can be halted or regeneration can even be initiated. Examples of this are factors for tissue regeneration which can be used e.g., in growth disorders, in degenerative diseases such as osteoporosis, arthritis or impaired wound healing. Here, the present system offers not only the advantage that the protein can be provided selectively and in the correct dosage but in addition it is possible to provide the protein in a certain time window. Thus, for example, with impaired wound healing, the relevant factor can be provided for a limited time by dosed administration of the RNA molecule. In addition, it can be arranged that the RNA is selectively brought to the site of its desired action.

“Effective amount” of a nucleic acid molecule refers to an amount sufficient to exert a therapeutic effect. In one embodiment, the term refers to an amount sufficient to elicit expression of a detectable amount of the recombinant protein.

### Exemplary Non-Viral Delivery Vehicles

In one embodiment, a non-viral delivery vehicle comprises inorganic nanoparticles, e.g., calcium phosphate or silica particles; polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM.

In one embodiment, the delivery vehicle comprises a cationic lipid, e.g., N-[1-(2,3-dioleoyloxy) propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, no delivery vehicle is employed, e.g., naked cmRNA is employed alone or with a scaffold.

In one embodiment, physical methods including but not limited to electroporation, sonoporation, magnetoporation, ultrasound or needle injection may be employed to introduce naked cmRNA, complexes of cmRNA and a delivery vehicle or cmRNA encapsulated in particles, or a scaffold having complexes of cmRNA and a delivery vehicle or cmRNA encapsulated in particles, into a tissue.

### Exemplary Scaffolds

Exemplary properties of a scaffold for use in tissue engineering include at least one of the following: (i) Biocompatibility. After implantation, the scaffold or tissue engineered construct does not elicit an immune response or elicits a negligible immune reaction. (ii) Biogradability. A biodegradable scaffold allows for regeneration of tissue at the site of the implant. (iii) Mechanical properties. The scaffold has mechanical properties consistent with the anatomical site into which it is to be implanted. For example, bone or cartilage scaffold must have sufficient mechanical integrity to function from the time of implantation to the completion of the remodeling process. (iv) Scaffold architecture. Scaffolds may have an interconnected pore structure and/or high porosity.

Three individual groups of biomaterials, e.g., ceramics, synthetic polymers and natural polymers, are commonly used in the fabrication of scaffolds for tissue engineering. Although not generally used for soft tissue regeneration, there has been widespread use of ceramic scaffolds, such as hydroxyapatite (HA) and tri-calcium phosphate (TCP), for bone regeneration applications. Ceramic scaffolds are typically characterized by high mechanical stiffness, very low elasticity, and a hard brittle surface. From a bone perspective, they exhibit excellent biocompatibility due to their chemical and structural similarity to the mineral phase of the native bone. The interactions of osteogenic cells with ceramics are important for bone regeneration as ceramics are known to enhance osteoblast differentiation and proliferation.

Numerous synthetic polymers have been used including polystyrene, poly-1-lactic acid (PLLA), polyglycolic acid (PGA) and poly-dl-lactic-co-glycolic acid (PLGA).

A third commonly used approach is the use of biological materials as scaffold biomaterials. Biological materials such as collagen, various proteoglycans, alginate-based substrates and chitosan have all been used in the production of scaffolds for tissue engineering. Unlike synthetic polymer-based scaffolds, natural polymers are biologically active and typically promote excellent cell adhesion and growth. Furthermore, the natural polymers are also biodegradable and so allow host cells, over time, to produce their own extracellular matrix.

Collagen and collagen-GAG (CG) scaffolds may be altered through physical and chemical cross-linking. Collagen-hydroxyapatite (CHA) scaffolds, collagen-hydroxy apitite (CHA) scaffolds may be useful for bone defects. Suitable biocompatible materials for the polymers include but are not limited to polyacetic or polyglycolic acid and derivatives thereof, polyorthoesters, polyesters, polyurethanes, polyamino acids such as polylysine, lactic/glycolic acid copolymers, polyanhydrides and ion exchange resins such as sulfonated polytetrafluorethylene, polydimethyl siloxanes (silicone rubber) or combinations thereof.

In one embodiment, the scaffold polymer is formed from natural proteins or materials which may be crosslinked using a crosslinking agent such as 1-ethyl-3-(3-dimethylamino-propyl) carbodiimide hydrochloride. Such natural materials include albumin, collagen, fibrin, alginate, extracellular matrix (ECM), e.g., xenogeneic ECM, hyaluronan, chitosan, gelatin, keratin, potato starch hydrolyzed for use in electrophoresis, and agar-agar (agarose), or other “isolated materials”. An “isolated” material has been separated from at least one contaminant structure with which it is normally associated in its natural state such as in an organism or in an in vitro cultured cell population.

Other biocompatible materials include synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[α(4-aminobutyl)]-1-glycolic acid, polyethylene oxide (Roy et al., 2003), polyorthoesters (Heller et al., 2002), silk-elastin-like polymers (Megeld et al., 2002), alginate (Wee et al., 1998), EVAc (poly(ethylene-co-vinyl acetate), microspheres such as poly(D. L-lactide-co-glycolide) copolymer and poly(L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, an agarose hydrogel, or a lipid microtubule-hydrogel.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol (D,L-lactide-coglycolide), poly(lactic acid colysine) and polycaprolactone.

In one embodiment, the biocompatible material for the distinct polymer is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer employed as a scaffold may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Exemplary Lipids

In certain embodiments, one or more lipids in a delivery vehicle include one or more phosphatidyl-cholines (PCs) selected from 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), e.g., in a lipid mixture comprising between about 0.5% to about 20% or about 1% to about 10%, or about 5% to about 15%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] or DOTAP [18:1]; cholesterol between about 10% to about 30% or about 25% to about 35%, or about 30% to about 40%, and/or a PEG modified lipid such as PEG-C-DMA (dimyristroylpropyl-3 amine) between about 1% to about 3%, about 1.2% to about 2.5%, or about 1.3% to about 2%.

In certain embodiments, the one or more lipids may include phospholipid, a phosphatidyl-choline, a phosphatidyl-serine, a phosphatidyl-diethanolamine, a phosphatidylinosite, a sphingolipid, or an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the one or more lipids are comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the one or more lipids are comprised of phospholipid selected from a monoacyl or diacylphosphoglyceride.

In still other illustrative embodiments, the one or more lipids are comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the one or more lipids are comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

Other embodiments include the one or more lipids selected from 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(l′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000] (18:1 PEG-2000 PE), 1,2-dipalmitoyl-sn-glycero-3-phosphoethanolamine-N-[methoxy (polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid comprises a cationic lipid and optionally one or more zwitterionic phospholipids.

In one embodiment, lipids include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy (polyethylene glycol)-2000] (18:1 PEG-2000 PE), 1,2-dipalmitoyl-sn-glycero-3-phosphoethanolamine-N-[methoxy (polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid complexes.

Pegylated phospholipids maybe employed in the lipid complexes or nanoparticles, including for example, pegylated 1,2-distearoyl-sn-glycero-3-phosphoethanolamine (PEG-DSPE), pegylated 1,2-dioleoyl-sn-glycero-3-phosphoethanolamine (PEG-DOPE), pegylated 1,2-dipalmitoyl-sn-glycero-3-phosphoethanolamine (PEG-DPPE), PEG-C-DMA, and/or pegylated 1,2-dimyristoyl-sn-glycero-3-phosphoethanolamine (PEG-DMPE), among others, including a pegylated ceramide (e.g. N-octanoyl-sphingosine-1-succinylmethoxy-PEG or N-palmitoyl-sphingosine-1-succinylmethoxy-PEG, among others). The PEG generally ranges in size (average molecular weight for the PEG group) from about 350-7500, about 350-5000, about 500-2500, about 1000-2000. Pegylated phospholipids may comprise a portion of the lipid complexes or nanoparticles, e.g., they may comprise a minor component, or be absent. Accordingly, the percent by weight of a pegylated phospholipid in the complexes or nanoparticles ranges from 0% to 100% or 0.01% to 99%, e.g., about 0.5%, 1.0%, 1.5%, 2.0%, 2.5%, 3.0%, 3.5%, 4.0%, 5%, 10%, 15%, 20%, 25%, 30%, 35%, 40%, 50%, 55%, 60% and the remaining portion comprising at least one, two or three other lipid molecules, such as cholesterol, usually in amounts less than about 50% by weight, and one or more cationic lipids, usually in amounts less than about 60% by weight.

If the delivery vehicle comprises two or more distinct lipids, one of the lipids may be cationic, e.g., DOTAP, and at least one of the others is non-cationic, e.g., DPPC or DSPC. Ratios of the two or more distinct lipids can vary, for example, for two distinct lipids, the ratio of a non-cationic lipid, e.g., neutral lipid, to the cationic lipid may be 1:x wherein x>1, e.g., 2 or 2.5 or 3, or x=1.

The delivery vehicle may be formed from a single type of lipid, or a combination of two or more distinct lipids. For instance, one combination may include a cationic lipid and a neutral lipid, or a cationic lipid and a non-cationic lipid. Exemplary lipids for use in the cationic liposomes include but are not limited to DOTAP, DODAP, DDAB, DOTMA, MVL5, DPPC, DSPC, DOPE, DPOC, POPC, or any combination thereof. In one embodiment, the cationic liposome has one or more of the following lipids or precursors thereof:

Other lipids include N-[1-(2,3-dioleyloxy) propyl]-N,N,N-trimethylammonium chloride with a monovalent cationic head; N,N-dioctadecyl-N-4,8-diaza-10-aminodecanoyl glycine amide; 1,4,7,10-tetraazacyclododecane cyclen; imidazolium-containing cationic lipid having different hydrophobic regions (e.g., cholesterol and diosgenin); 1,2-dioleoyl-sn-glycero-3-phosphoethanolamine (DOPE); 3β-[N—(N′,N′-dimethylamino-ethane) carbamoyl) cholesterol (DC-Chol) and DOPE; O,O′-ditetradecanoyl-N-(α-trimethyl ammonioacetyl) diethanol-amine chloride, DOPE and cholesterol, phosphatidylcholine; 1,2-dilinoleyl-4-(2-dimethylaminoethyl)-[1,3]-dioxolane, 1,2-distearoyl-sn-glycerol-3-phosphocholine (DSPC) and cholesterol, 1-palmitoyl-2-oleoyl-sn-glycero-3-phosphocholine, DOPE, and 1,2-distearoyl-sn-glycero-3-phosphoethanolamine-N-(methoxy[polyethylene glycol-2000), 1,2-di-O-octadecenyl-3-trimethylammonium propane, cholesterol, and D-α-toco; 1,2-dioleoyl-3-trimethylammonium-propane, cholesterol; 3-β(N—(N′,N′-dimethyl, N′-hydroxyethyl amino-propane) carbamoyl) cholesterol iodide, DMHAPC-Chol and DOPE in equimolar proportion, or 1-palmitoyl-2-oleoyl-sn-glycero-3-ethylphosphocholine: cholesterol, dimethyldioctadecylammonium (DDAB); 1,2-di-O-octadecenyl-3-trimethylammonium propane; N1-[2-((1S)-1-{(3-aminopropyl)amino]-4-[di(3-amino-propyl)amino)amino]butylcarboxamido)ethyl]-3,4-di[oleyloxy]-benzamide (MVL5); 1,2-dioleoyl-3-dimethylammonium-propane (DODAP); 1,2-di-O-octadecenyl-3-trimethylammonium propane (DOTMA); 1,2-dioleoyl-sn-glycero-3-phosphocholine (DOPC); 1-palmitoyl-2-oleoyl-sn-glycero-3-phosphocholine (POPC).

### Exemplary Formulations, Dosages and Routes of Administration

The isolated nucleic acid, e.g., cmRNAs, can be formulated as pharmaceutical compositions and administered to a mammalian host, such as a human patient in a variety of forms adapted to the chosen route of administration, e.g., orally or parenterally, by intravenous, intramuscular, topical, local, or subcutaneous routes. In one embodiment, the composition having isolated polypeptide or peptide is administered to a site of bone loss or cartilage damage or is administered prophylactically.

In one embodiment, the isolated nucleic acid, e.g., cmRNAs, may be administered by infusion or injection. Solutions of the miRNA or its salts, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in complexes, liposomes, nanoparticles or microparticles. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In some cases, it may be desirable to include isotonic agents, for example, sugars, buffers or sodium chloride. Prolonged absorption of the injectable compositions can be brought about by the use in the compositions of agents delaying absorption, for example, microparticles, or aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze-drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the isolated nucleic acid, e.g., cmRNAs, can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the isolated nucleic acid, e.g., cmRNAs, or isolated polypeptide in a liquid composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the isolated nucleic acid, e.g., cmRNAs, for use alone or with other agents will vary with the route of administration, the nature of the condition being treated and the age and condition of the patient and will be ultimately at the discretion of the attendant physician or clinician.

The isolated nucleic acid, e.g., cmRNAs, or isolated polypeptide may be conveniently administered in unit dosage form; for example, containing 5 to 1000 mg, conveniently 10 to 750 mg, or conveniently 50 to 500 mg of active ingredient per unit dosage form.

In general, a suitable dose of nucleic acid or polypeptide may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

For viral vectors, the dose may be from about 1×10⁴ GC/kg, about 1×10⁵ GC/kg, about 1×10⁶ GC/kg, about 1×10⁷ GC/kg, about 1×10⁸ GC/kg, about 1×10⁹ GC/kg, about 1×10¹⁰ GC/kg, such as 1×10¹¹ GC/kg, 2×10¹¹ GC/kg, 3×10¹¹ GC/kg, 4×10¹¹ GC/kg, 5×10¹¹ GC/kg, 6×10¹¹ GC/kg, 7×10¹¹ GC/kg, 8×10¹¹ GC/kg, 9×10¹¹ GC/kg, or 1×10¹² GC/kg.

The ultimate dosage form may be sterile, fluid and stable under the conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The prevention of the action of microorganisms can be brought about by various antibacterial and antifungal agents, for example, parabens, chlorobutanol, phenol, sorbic acid, thimerosal, and the like. In many cases, it may be desirable to include isotonic agents, for example, sugars, buffers or sodium chloride.

Both local administration and systemic administration are contemplated. One or more suitable unit dosage forms can be administered by a variety of routes including local. The formulations may, where appropriate, be conveniently presented in discrete unit dosage forms and may be prepared by any of the methods well known to pharmacy. Such methods may include the step of bringing into association the subunit components, e.g., one or more lipids, subunits of a polymer or co-polymer, or the polymer or co-polymer, and the RNA and optionally liquid carriers, solid matrices, semi-solid carriers, finely divided solid carriers or combinations thereof, and then, if necessary, introducing or shaping the product into the desired delivery system.

The delivery vehicle such as a pharmaceutically acceptable carrier(s) may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The active agent may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

In one embodiment, the DNA or RNA may be formulated for administration, e.g., by injection, infusion, a pump or a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulary agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

The formulations and compositions described herein may also contain other ingredients such as antimicrobial agents or preservatives.

The pharmaceutical formulations can also take the form of an aqueous or anhydrous solution, e.g., a lyophilized formulation, or dispersion, or alternatively the form of an emulsion or suspension.

### Pharmaceutical Compositions

The disclosure provides a composition comprising, consisting essentially of, or consisting of microparticles, nanoparticles, liposomes or lipid complexes comprising nucleic acid encoding CST6 or a structural and/or functionally related polypeptide, and optionally a pharmaceutically acceptable (e.g., physiologically acceptable) carrier. In one embodiment, additional components can be included that do not materially affect the composition (e.g., adjuvants, buffers, stabilizers, anti-inflammatory agents, solubilizers, preservatives, etc.). In one embodiment, when the composition consists of the nucleic acid and the delivery vehicle and optionally a scaffold or other pharmaceutically acceptable carrier, the composition does not comprise any additional components. Any suitable carrier can be used, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the therapeutic nucleic acid is administered in a composition formulated to protect the therapeutic nucleic acid from damage prior to administration. In addition, one of ordinary skill in the art will appreciate that the therapeutic nucleic acid can be present in a composition with other therapeutic or biologically-active agents.

Injectable depot forms are envisioned including those having biodegradable polymers such as polylactide-polyglycolide. Depending on the ratio of inhibitor to polymer, and the nature of the particular polymer employed, the rate of inhibitor release can be controlled. Examples of other biodegradable polymers include poly(orthoesters) and poly(anhydrides). Depot injectable formulations are also prepared by entrapping the nucleic acid optionally in a complex with a delivery vehicle in liposomes or other lipid complexes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho) esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the nucleic acid in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the protease inhibitor to elicit a desired response in the individual. One of ordinary skill in the art can readily determine an appropriate protease inhibitor dose range to treat a patient having a particular disease or disorder, based on these and other factors that are well known in the art.

In one embodiment, the composition is administered once to the mammal. It is believed that a single administration of the composition may result in persistent expression in the mammal, optionally with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of the protease inhibitor, e.g., encoded by a cmRNA, plasmid or viral vector, e.g., an AAV or lentivirus vector.

### Exemplary Routes of Administration, Dosages and Dosage Forms

Administration of cmRNA, isolated DNA or RNA encoding CST6 or isolated CST6 polypeptide may be continuous or intermittent, depending, for example, upon the recipient's physiological condition, and other factors known to skilled practitioners. The administration of the therapeutic agent may be essentially continuous over a preselected period of time or may be in a series of spaced doses. Both local administration, e.g., to a site of a bone defect, and systemic administration are contemplated. Any route of administration may be employed, e.g., intravenous, intranasal or intrabronchial, or local administration. In one embodiment, compositions may be subcutaneously, orally or intravascularly delivered.

One or more suitable unit dosage forms comprising the cmRNA, isolated DNA or RNA encoding CST6 or isolated CST6 polypeptide, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the therapeutic nucleic acid with liquid carriers, solid matrices, semi-solid carriers, finely divided solid carriers or combinations thereof, and then, if necessary, introducing or shaping the product into the desired delivery system.

The amount of the cmRNA, isolated DNA or RNA encoding CST6 or isolated CST6 polypeptide administered to achieve a particular outcome will vary depending on various factors including, but not limited to the condition, patient specific parameters, e.g., height, weight and age, and whether prevention or treatment, is to be achieved.

The cmRNA, isolated DNA or RNA encoding CST6 or isolated CST6 polypeptide may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The complexes or particles containing one or more cmRNA, isolated DNA or RNA encoding CST6 or isolated CST6 polypeptide may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The cmRNA, isolated DNA or RNA encoding CST6 or isolated CST6 polypeptide can be provided in a dosage form containing an amount effective in one or multiple doses. The therapeutic nucleic acid may be administered in dosages of at least about 0.0001 mg/kg to about 20 mg/kg, of at least about 0.001 mg/kg to about 0.5 mg/kg, at least about 0.01 mg/kg to about 0.25 mg/kg, at least about 0.1 mg/kg to about 0.25 mg/kg of body weight, about 0.1 mg/kg to about 0.5 mg/kg, about 0.5 mg/kg to about 2 mg/kg, about 1 mg/kg to about 5 mg/kg, about 5 mg/kg to about 10 mg/kg, or about 10 mg/kg to about 20 mg/kg although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of therapeutic nucleic acid can be administered.

Pharmaceutical formulations containing the cmRNA, isolated DNA or RNA encoding CST6 or isolated CST6 polypeptide can be prepared by procedures known in the art using well known and readily available ingredients. For example, the agent can be formulated with common excipients, diluents, or carriers, and formed into tablets, capsules, suspensions, powders, and the like. The cmRNA, isolated DNA or RNA encoding CST6 or isolated CST6 polypeptide containing particles or complexes can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the cmRNA, isolated DNA or RNA encoding CST6 or isolated CST6 polypeptide containing particles or complexes may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the cmRNA, isolated DNA or RNA encoding CST6 or isolated CST6 polypeptide composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the cmRNA, isolated DNA or RNA encoding CST6 or isolated CST6 polypeptide composition may be administered via nose drops, a liquid spray, such as via a plastic bottle atomizer or metered-dose inhaler. Typical of atomizers are the Mistometer (Wintrop) and the Medihaler (Riker).

The local delivery of the cmRNA, isolated DNA or RNA encoding CST6 or isolated CST6 polypeptide composition can also be by a variety of techniques which administer the therapeutic nucleic acid composition at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals include all vertebrates, e.g., mammals and non-mammals, such as non-human primates, sheep, dogs, cats, cows, horses, chickens, amphibians, and reptiles, although mammals, such as non-human primates, sheep, dogs, cats, cows and horses, are envisioned. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape

The invention will be described by the following non-limiting examples.

### Example 1

Fabricate and characterize a biomaterial-based cmRNA (CST6) delivery system. Incorporating active agents such as cmRNA (CST6) within a well-established scaffold for bone regeneration such as a CM, can lead to a highly predictable and effective scaffold for bone regeneration applications. Thus, stable nucleic acid lipid particles (SNALP)-CST6 (cmRNA) nanoparticles (CST6 NPs) were synthesized and characterized their biocompatibility, transfection efficacy and functionality of transfection with CST6 NPs in bone marrow-derived stem/stromal cells (BMSC), murine pre-osteoblastic cells (MC3T3-E1), RAW264.7 and bone marrow-derived macrophages (BMM) were tested using molecular and colorimetric assays. The osteogenic differentiation and osteoclast inhibition of cmRNA (CST6) treated cells is determined by evaluating the expression of specific genes at specific time points, post-treatment (Laird et al., 2021; Elangovan et al., 2014; Atluri et al., 2015; D'Angelo et al., 2020; Lee et al., 2016; Guo et al., 2020; Yeon et al., 2019; Wang et al., 2019; D. Mello et al., 2017; Chakka et al., 2021). The mineralization ability of cmRNA (CST6) on BMSC and MC3T3-E1 cells is assessed using Alizarin red staining and an alkaline phosphatase (ALP) assay. In addition, inhibition of osteoclast maturation is evaluated using tartrate-resistant acid phosphatase (TRAP) staining. The attachment and proliferation of BMSCs on the scaffolds containing cmRNA (CST6) is also assessed using imaging techniques.

Synthesis of cmRNA (CST6)

In vitro transcription of cmRNA (CST6) wis performed using plasmid constructs containing complementary DNA (cDNA) encoding CST6 as the template, in a similar manner to the protocol described in Elangovan et al., 2015, and Khorsand et al., 2017. This cDNA is flanked upstream by a T7 promoter and downstream by a polyA tail of 120 bp in length. Plasmids are first linearized with Xba-I, following which, their purity is verified and quantified spectrophotometrically. Using commercially available high yield transcription kits, cmRNA (CST6) is synthesized and capped with the anti-reverse cap analog (ARCA; 7-methyl (3′-O-methyl) GpppGm7G (5′) ppp (5′) G). To achieve mRNA modification, the following modified ribonucleic acid triphosphates are added to the reaction at a ratio of 10%:2-thiouridine-5′-triphosphate and 5-methylcytidine-5′-triphosphate. Synthesized cmRNA is purified and analyzed for size and purity. Once the cmRNA (CST6) is synthesized, its immunogenicity is evaluated. The binding of cmRNA to pattern recognition receptors such as toll-like receptors (TLR) 3, 7 and 8 is determined in peripheral blood mononuclear cells using RNA immunoprecipitation as described previously (Elangovan et al., 2015; Elangovan et al., 2014). Scrambled cmRNA (CST6) (e.g., altered sequence) is also synthesized and used in in vitro and in vivo experiments as a negative control.

### Synthesis and Characterization of SNALP-cmRNA (CST6) Nanoparticles (CST6 NPs)

cmRNA wis encapsulated in SNALP using a controlled step-wise dilution method. The lipid components of the SNALP are cationic lipid (Selleck Chem), dipalmitoylphosphatidylcholine (DPPC; Avanti Polar Lipids), synthetic cholesterol (Sigma) and PEG-C-DMA used at a molar ratio of 57:7:34:1.5. Upon formation of the loaded particles, SNALP are dialyzed against phosphate based saline and filter sterilized through a 0.2 μm filter before use. After a series of studies, SNALPs loaded with cmRNA had mean particle sizes of 81-92 nm and 87-96% of the cmRNA was encapsulated within the lipid particles. The final cmRNA/lipid ratio in formulations used for in vivo testing is approximately 0.15 (wt/wt). After the synthesis of CST6 NPs, the size and polydispersity of the synthesized SNALPs is determined using dynamic light scattering and transmission electron microscopy (TEM) analyses. SNALPs are known for their spherical, monodisperse features. Zeta potential (surface charge) measurements are determined using the Zetasizer Nano ZS. Once the physical characteristics are assessed, encapsulation efficiency and cmRNA loading within SNALPs is elucidated using high-performance liquid chromatography (HPLC), spectrophotometry and gel electrophoresis, respectively. The physico-chemical characterization is carried out before and after lyophilization to determine the effect (if any) of freeze thaw cycles on formulation integrity and lyoprotectants are included should the need arise (Laird et al., 2020). Mechanical integrity and properties of the delivery system are analyzed by measuring the compressive and tensile strength of the CM before and after loading of CST6 NP. CST6 NPs are tested for toxicity in BMSC, MC3T3-E1, RAW264.7 and BMM cells using MTS, LDH, Caspase 3/9 and ROS assays (Acri et al., 2019; Wongrakpanich et al., 2016; Areecheewakul et al., 2020; Wang et al., 2020). Toxicity is tested after 4, 24 and 48 hours (hrs) incubation under serum-free and serum-containing conditions. The amount of CST6 secreted by transfected cells into the cell culture medium is determined using ELISA. Untreated cells are a separate group to obtain baseline CST6 secretion levels by these cells at defined time points. The formulation of cmRNA loaded SNALPs described above showed that cmRNA loaded SNALPs (LD 50 value=173+/−12 μg/ml) have significantly lower toxicity than polyethylenimine (PEI) (LD50 value=25+/−μg/ml). One μg/μl of CST6 NP encoding for green fluorescent protein (GFP) generated a mean 67% GFP positive cells relative to a mean 47% GFP positive cells from 1 μg/μl PEI-cmRNA (N/P ratio of 10) polyplexes in BMSCs. Thus, SNALPs are less toxic and more effective at delivering cmRNA to cells than PEI.

### Measuring Osteogenic Differentiating Activity of SNALP-cmRNA (CST6) NPs in Human BMSC and MC3T3-E1 Cells

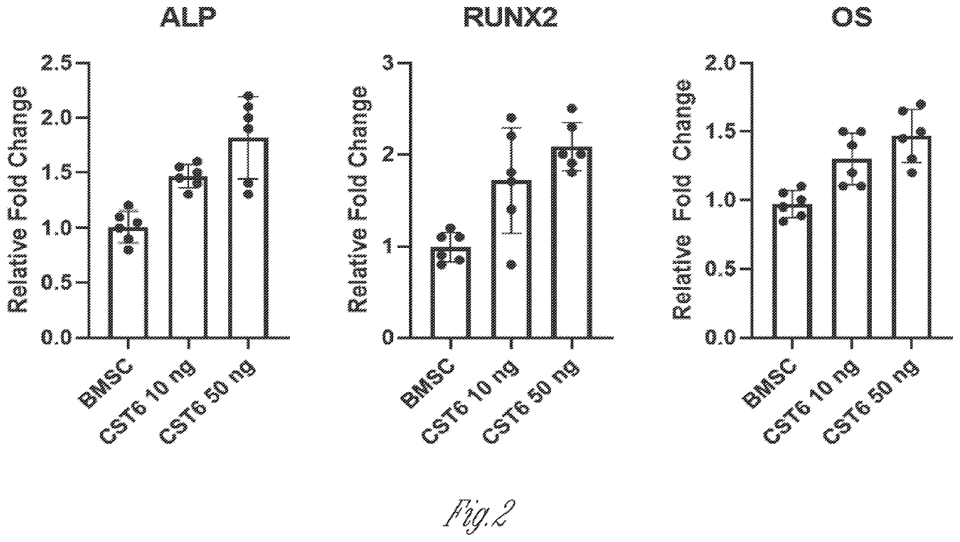

Human BMSCs and MC3T3-E1 cells are treated with SNALP alone, cmRNA (CST6) alone, recombinant human CST6 (rCST6), PEI-pDNA (encoding for CST6) polyplexes, SNALP-scrambled cmRNA (CST6) NPs and SNALP-cmRNA (CST6) NPs in serum-free media. PEI is being used to deliver pDNA because in contrast to the cmRNA, pDNA did not load efficiently into the SNALPs. Additional controls include BMP-2 protein, cmRNA encoding BMP-2 loaded in SNALP and PEI-pDNA (encoding BMP-2) polyplexes with methods and protocols for preparing these described in Elangovan et al., Atluri et al., 2015; Acri et al., 2019b; Khorsand et al., 2019; Acri et al., 2020; Khorsand et al., 2020; Elangovan et al., 2015; Khorsand et al., 2017. The cells are lysed at 12, 24, 48, 72 and 96 hours and the expression of bone specific genes (collagen type I, RUNX2, ALP, VEGF, osteocalcin, Cbfa-1 and osterix) is determined in the total extracted RNA using reverse transcriptase polymerase chain reaction (RT-PCR). The ratio of PCR product intensity to β-actin (internal control) intensity is calculated in the scanned gel using image analysis software as described previously (Elangovan et al., 2013). In addition, ALP activity in the transfected BMSCs is determined by lysing the cells transfected with the same preparations, 48 and 96 hours post-transfection and measuring the enzyme activity using the ALP reagent containing p-nitrophenylphosphate at 405 nm as described previously (Jono et al., 1998). CST6 NPs are also tested for tube formation properties in human umbilical vascular endothelial cells (HUVECs) using a protocol described previously and to determine the vascularization (angiogenesis) properties of CST6 NPs (Chakka et al., 2021). To further evaluate the functionality of transfection, 2 and 3 weeks post-transfection with the six preparations as described above [in media containing ascorbic acid (50 μg/ml) and b-glycerophosphate (10 mM)], the cells are fixed at 4° C. in 70% ethanol and stained with Alizarin red S stain (40 mM, pH 4.2) for 10 minutes and imaged for calcific deposits as described previously (Chakka et al., 2021; Atluri et al., 2017). Once the transfection efficiency of CST6 NPs is determined, the attachment and proliferation of BMSCs and MC3T3-E1 cells on CM with and without CST6 NPs are evaluated using scanning electron microscopy (SEM) and confocal microscopy as described previously (Elangovan et al., 2014). Preliminary studies have shown that CST6 protein induces osteogenic differentiation in MC3T3-E1 cells and upregulates osteogenic genes (ALP, RUNX2 and OS) in BMSCs (FIG. 2).

### Measuring Osteoclast Differentiation Inhibition Activity of SNALP-cmRNA (CST6) NPs in Macrophages

Reduction of osteoclast differentiation are monitored by evaluation of Tartrate-resistant acid phosphatase (TRAP) staining and RT-PCR analysis for NTATc1, TRAP, MMP-9, OSCAR, DC-STAMP, ATP6vOd2 and Cathepsin K genes using methods and protocols that have been described previously (D'Angelo et al., 2020; Lee et al., 2016; Guo et al., 2020; Yeon et al., 2019; Wang et al., 2019). Preliminary studies showed CST6 protein inhibits osteoclast differentiation in TRAP staining.

### Results

CST6 NPs stimulate osteogenic differentiation in BMSCs and MC3T3-E1 cells and inhibit osteoclast maturation in RAW264.7 cells and BMM. RNA immunoprecipitation confirms the ameliorated immunogenicity of cmRNA (CST6). SNALP-cmRNA (CST6) shows low toxicity and high transfection efficiency. Expression levels of bone specific genes are t significantly higher in cells transfected with cmRNA (CST6), compared to other groups. The Alizarin red S staining experiment, the ALP assay and the RT-PCR experiments validate the functionality of transfection of cmRNA (CST6) in all cells. Osteogenic supplements such as ascorbic acid or β-glycerophosphate may be added to facilitate differentiation. Alternative vectors such as polyamidoamine (PAMAM) dendrimers or hybrid PLGA/PEI nanoparticles may also be used as a delivery vehicle using methods and protocols described previously (Zhang et al., 2007; Zhang et al., 2008; Intra & Salem, 2010).

Determine the In Vivo Efficacy of a Biomaterial-Based cmRNA (CST6) Delivery System to Induce Bone Formation

After in vitro characterization of the cmRNA (CST6) delivery system in BMSC, MC3T3-E1, RAW264.7 cells and BMM, the in vivo efficacy of the system is evaluated in a standardized unilateral diaphyseal femoral defect (6 mm) in Sprague Dawley rats stabilized with a commercially available plating system which provides a defect that will not otherwise heal in 12 weeks. The delivery system is first evaluated in unilateral diaphyseal femoral defects to identify the effective dose (ED) of cmRNA (CST6) using the following groups: CM only, CM with SNALP, CM containing CST6 NPs in the following cmRNA doses: 25, 50, 75 and 100 μg (n=16 per group). These groups are analyzed using standard radiographs (caudocranial and lateral views), mechanical testing and gait analysis (Catwalk_XT) at time points 1, 2, 4, 6, 8 and 12 weeks. In addition, gene and protein markers for osteoclast maturation and osteogenic differentiation are measured at week 12. Systemic toxicity and biodistribution of Cy5.5. labelled cmRNA NPs loaded in CMs are tested 24 hours and 12 weeks after implantation. Bone formation is assessed in the unilateral diaphyseal femoral defects in rats using the following groups (Table 2): 1) defects left empty, 2) defects treated with CM alone, 3) defects treated with CM containing SNALP, 4) defects treated with CM containing ED of recombinant protein form of CTS6, 5) defects treated with CM containing ED of PEI-pDNA, 6) defects treated with CM containing ED of SNALP-scrambled cmRNA (CST6), 7) defects treated with CM containing CST6 (ED equivalent dose) with SNALP-cmRNA (CST6), and 8) defects treated with CM containing 25 μg BMP-2 protein. Each group will have n=16 per group. All characterization used in determining the effective dose including micro-CT (μCT), RT-PCR analysis, gait analysis and mechanical testing is carried out for these groups.

TABLE 2
Bone Defect Model Experimental Groups

| | |
|---|---|
| 1 | Empty Defect (n = 16) |
| 3 | Defects containing CM (n = 16) |
| 3 | Defects containing CM with SNALP (n = 16) |
| 4 | Defects containing CM with ED equivalent dose |
| | of CST6 recombinant protein (n = 16) |
| 5 | Defects containing CM with ED equivalent |
| | dose of PEI-pDNA (CST6) (n = 16) |
| 6 | Defects containing CM with ED of |
| | SNALP-scrambled (CST6) (n = 16) |
| 7 | Defects containing CM with ED of |
| | SNALP-cmRNA (CST6) (n = 16) |
| 8 | Defects containing CM with BMP-2 protein (n = 16) |

16 defects per group CM: collagen matrix and ED: effective dose

### Surgical Procedures

Approach (Based on RISystems (Davos, Switzerland), RatFix Surgical Technique Guide and Team Experience): Male and female rats receive a dose of enrofloxacin (5 mg/kg, SC) and Buprenorphine SR (1-1.2 mg/kg, subcutaneous (SC)) pre-operation (pre-op). Rats are anesthetized with Isoflurane (1-5%) delivered in oxygen via a nose cone mask. Rats are surgically prepped with chlorhexidine scrub and alcohol (alternating 3 times each) and chlorhexidine solution prior to surgery at each site. Following prep, Bupivacaine 0.5% solution (0.1-0.2 mg/kg) are injected at the surgical site for local analgesia prior to skin incision. A longitudinal skin incision along the lateral aspect of the femur from the hip to the stifle. A small incision is made through the fascia lata and blunt elevation of the vastus lateralis and biceps femoris muscles which are split and retracted with the tensor fasciae latae muscle to expose the full length of the femur preserving the sciatic nerve.

Plate Application: The plate is placed on the exposed femur in craniolateral direction by externally rotating the femur. The plate is fixed to the bone with forceps in the middle of the diaphysis. The first screw is used to align the plate. The first hole is drilled distal to the planned fracture gap and the screw inserted. The hole for the second screw is drilled proximal to the planned osteotomy. The remaining screws are placed and tightened.

Defect Creation: Placement of the saw guide over the shafts of the screws nearest to the osteotomy sites and a 6 mm bone defect is created by using a Gigli saw. The bone segment and guide are removed.

Implantation: Appropriate pre-sized implants are placed into the diaphyseal defect.

Wound Closure: The vastus lateralis is repositioned loosely over the plate. The fascia is closed with a 4-0 Vicryl suture followed by the skin. Recovery: Rats will be recovered on a warming blanket and under a heat lamp until rats are able to sit sternal and ambulate. Rats may receive 1-4 ml of fluids (saline or lactated ringers) SC prior to recovery. Rats will be monitored for health and wellbeing as described in the vertebrate animal section.

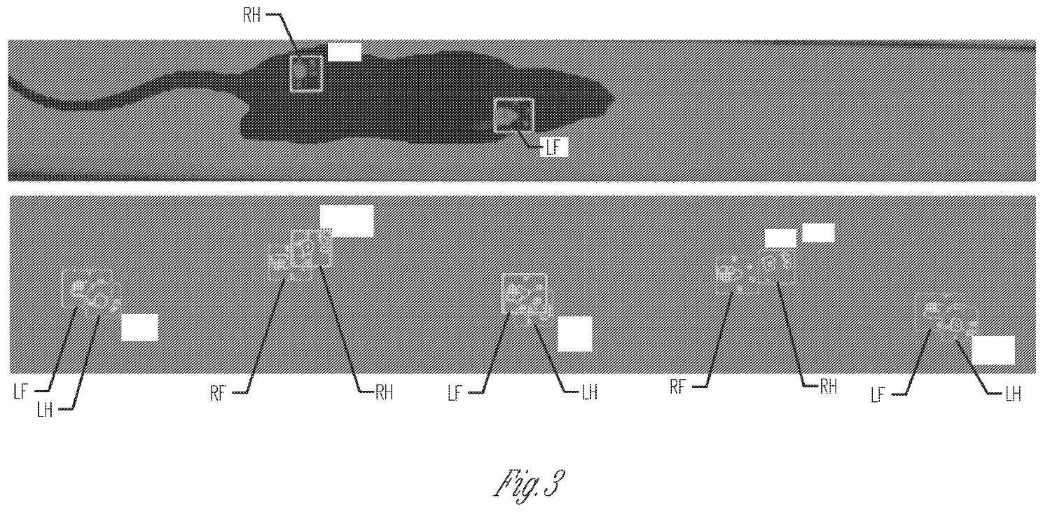

Biodistribution and systemic toxicity. These studies are carried out on (n=4 per time-point) rats 24 hours after implantation and 12 weeks after implantation for CM alone and CST6 NP loaded CM. These studies include evaluation of liver enzymes, Cy5.5 conjugated cmRNA concentrations in heart, lung, liver, and kidneys and histology of all the aforementioned organs using methods and protocols described previously (Ebeid et al., 2017; Morris et al., 2017; Ebied et al., 2018; Skeie et al., 2020; Naguib et al., 2021).
Gait Analysis. Gait analysis is conducted using the CatWalk XT system (Version 10.6; Noldus Information Technology, The Netherlands). This automated system includes an enclosed illuminated glass walkway that rodents traverse. A camera captures each individual footprint, which allows measurement of both dynamic and static gait parameters as demonstrated in FIG. 3.

Rats are trained prior to surgery for 1-2 weeks to acclimate them to the test setup. The rats walk/run freely though the enclosed glass walkway to the end where their cage-mate is waiting in a goal-cage. The cage-mate is motivation for the rat to traverse the walkway. If needed, food rewards may be used for motivation/reward as well. The goal of the training is for the rats to perform five successful runs, where a successful run is a run duration between 1-5 seconds, no explicit stops, a velocity variation no more that 70%, and a maximum velocity of no more than 400 mm/s.

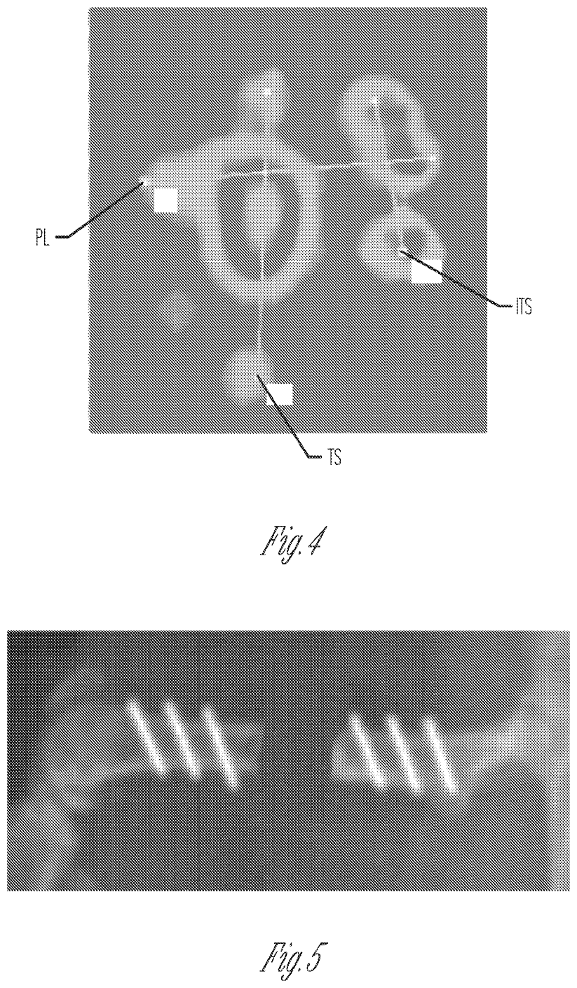

On the day prior to surgery, five successful baseline runs are performed by each rat. Post-surgery measurements are taken 1, 2, 4, 6, 8, and 12 weeks. Each time point needs five successful trials. During the off weeks, rats are placed on CatWalk to maintain training and consistency, but trials are not recorded. CatWalk software is used to ‘auto classify’ pawprints. However, manual inspection and editing occur according to the systematic manual classification method described by Chen and colleagues (2017). To assess pain and healing, several parameters are calculated from the successful registered gait data: 1) Intensity, 2) Print area, 3) Stand duration, 4) Duty cycle, 5) Manual Print Length and 6) Manual Toe spread and intermediate toe spread. This are included when pawprints allow for measurement as demonstrated in FIG. 4.

The ratio between the left and right hind limb parameters is calculated for all trials. The pre-operative trial is the baseline trial and all post-op trials are standardized against the baseline. The right to left limb ratio considers the variance due to weight and run calibration, whereas the standardization to baseline ratios accounts for the natural tendency of each rat to bear weight on a particular side (Chen et al., 2017).

### Plain Radiographs

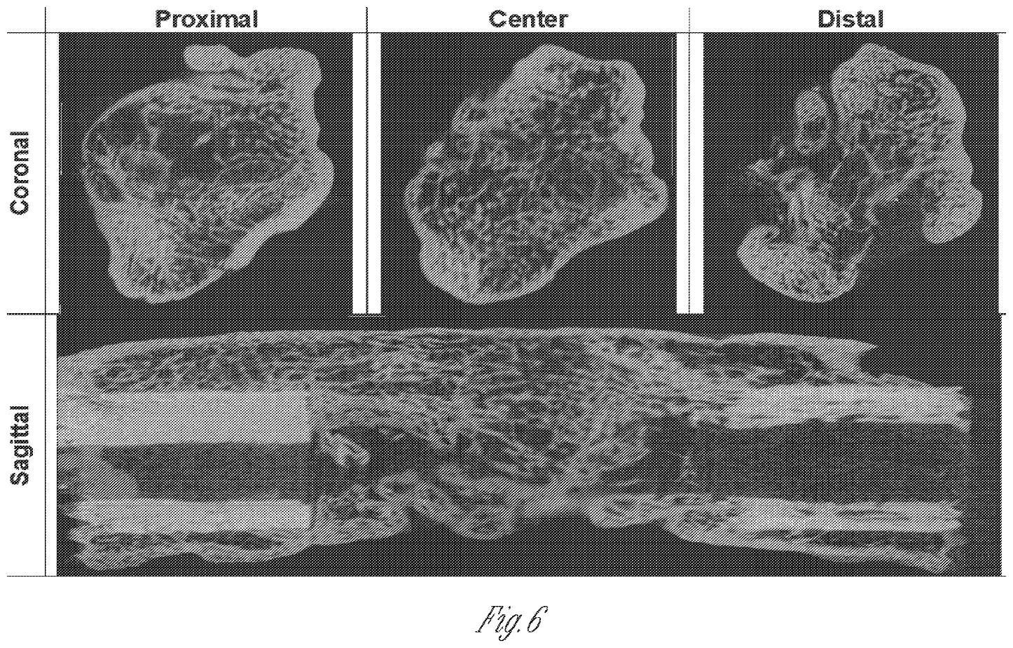

Standard craniocaudal and oblique/lateral radiographs are obtained from each femur using a digital x-ray unit as demonstrated in FIG. 5. The plain radiograph films are assessed for signs of graft migration, osteolysis, fracture, and/or any other adverse event that may be present. Radiographic evaluation are performed blindly by three reviewers with experience interpreting radiographs. Estimated bone formation within the defect, as well as the timing of defect-bridging and corticalization are scored. Healing is scored by assessing the number of cortices bridged in the 2 radiographic views (craniocaudal and oblique/lateral). Each view has two possible numbers of bridging cortices. A total score of 3 or 4 indicates radiographic healing (Lack et al., 2013).

### μCT

At time of euthanasia, images are obtained from each implant site with resolution of 9 voxels and reconstructed for three-dimensional analysis with subsequent thresholding and software image analysis. Total calcified bone volume, density, porosity, trabecular number, and total bone surface area are measured as demonstrated in FIG. 6.

### RNA Extraction and RT-PCR

At week 12, biopsies are taken and stored in RNAlater™ Stabilization Solution. Total RNA is extracted using a commercially-available kit. The integrity of the purified RNA is assessed, and then is reverse transcribed into cDNA which is used as the template for RT-PCR. Expression of genes of interest are investigated using RT-PCR using appropriate primers, probes, and internal controls; each sample is run in duplicate (CST-6, RUNX2, OS, Cbfa-1, osterix, BMP-2, 4, 6, 7, VEGF, NTATc1, TRAP, OSCAR, DC-STAMP, ATP6vOd2, Cathepsin K, FGF-2, TGF-β, ALK-2, and ALK-3). Quantitation is performed using the 2^{-ΔΔCt} method with expression levels of target genes normalized to the expression of internal control genes.

### Histomorphometry

Static bone histomorphometric analysis is performed on one section from each defect using Olympus Cell Sense Dimension software (Olympus Life Sciences) following standard SOP's. At least six thin sections (two each obtained from one of three longitudinal planes across each defect) are stained with hematoxylin and eosin (H&E) and Massons' Trichrome for histopathological analysis. All sections are scored according to recommendations provided in ISO 10993-6, Annex E. The histomorphometry scorer is blinded as to the specimen identity when performing measurements. Measurements will be made in a region of interest (ROI) 10 mm length (centered over the length of the defect). Degree of vascularization is measured using protocols we have described previously (Chakka et al., 2021).

### Mechanical Testing

Structural stiffness and strength are determined by a torsion test to failure. When a structure is loaded in torsion, failure occurs at the weakest location along the length and the loading is independent of cross-sectional orientation. Mechanical testing is done with the plate removed. Bones are potted so the screw holes don't create stress concentrations for testing and μ-CT is used to relate cross section geometry to orientation during the test. To prepare the specimens for testing, the proximal and distal ends of the femurs are embedded in pieces of square PVC tubing with fast setting polymethylmethacrylate (Coralite Dental Powder and Fast Set Liquid). An appropriate spacer is used to determine the gauge length of the regenerated bone. Alignment of the two ends is maintained with a custom positioning fixture. All specimens are kept moist during preparation with saline. The femur is tested using a servohydraulic biaxial load frame (Mini-Bionix 858) in torsion at 1 degree/sec to failure. Measured torque and angle values are used to determine torsional stiffness, failure torque, and angular rotation at failure. The relationship between bone maturity and failure is graded using the scoring scheme described by White et al. (White III et al., 1977).

### Statistical Analysis

For in vitro assays, all the measurements are made in triplicate (n=8). All treatment and control groups are compared using one-way analysis of variance (ANOVA) and Tukey's post-hoc test. The primary outcome is percentage new bone volume derived from μCT analysis. Eight animals per group are used for this purpose. This sample size determination has ample power (>80%) detecting medium effect sizes for primary group comparison (Cohen's d>1.45, two sided test with significance level of 0.05).

### Results

The unilateral diaphyseal femoral defect model identifies the ED of cmRNA (CST6). Both in μCT and histology, complete bone fill and bridging of defects in group 7 is comparable to group 4 and superior to the rest of the tested groups. Markers of osteogenic differentiation are upregulated and markers of osteoclast differentiation inhibited with the CST6 NP loaded CM relative to other groups. CST6 NPs stay localized to the femoral defect and demonstrate minimal to no systemic toxicity. Any observed liver enzyme elevation above 3-fold is mitigated by a reduction in loading of CST6 NPs in CM.

### REFERENCES

Acri et al., J. Tissue Eng. Regen. Med., 13:2256 (2019a).
Acri et al., MRS Adv., 4:3505 (2019).
Acri et al., Tissue Eng. Part A, ______:______ (2020).
Annunziata et al., Cancer Cell., 12:115 (2007).
Areecheewakul et al., NanoImpact., 18: (2020).
Atluri et al., Int. J. Oral & Maxillof. Imp., 32: e83 (2017).
Atluri et al., Int. J. Oral Maxillofac. Implants, 32: e83 (2017).
Atluri et al., Mol. Pharm., 12:3032 (2015).
Bartel et al., Blood, 114:2068 (2009).
Bergsagel et al., Blood, 106:296 (2005).
Bird et al., Nat. Rev. Immunol., 9:871 (2009).
Boyce et al., Orthopedic Clinics, 30:571 (1999).
Brage et al., Bone, 34:412 (2004).
Broyl et al., Blood, 116:2543 (2010).
Chakka et al., RSC Advances, ______:______ (2021).
Chen et al., J. Neurosci. Methods, 283:92 (2017).
Cheng et al., J. Invest. Dermatol., 129:1232 (2009).
Cheng et al., J. Biol. Chem., 281:15893 (2006).
Cohen et al., Nat. Med., 27:491 (2021).
Colditz et al., J. Bone Miner. Res., 33:1698 (2018).
Coleman et al., Cancer Treat Rev., 36:615 (2010).
D'Angelo et al., Biomed. Pharmacother., 123:109764 (2020).
D'Costa et al., Oncotarget., 5:1609 (2014).
D'Mello et al., AAPS J., 19:43 (2017).
D'Mello et al., Arch. Oral Biol., 60:1749 (2015a).
D'Mello et al., Curr. Pharm. Biotechnol., 16:655 (2015b).
D'Mello et al., J. Tissue Eng. Regen. Med., 10:927 (2016).
de Jong et al., Nat. Immunol., 22:769 (2021).
Drake et al., Endocr. Rev., 38:325 (2017).
Ebeid et al., J. Chromatogr. B. Analyt. Technol. Biomed. Life Sci., 1044-1045:70 (2017).
Ebeid et al., Nat. Nanotechnol., 13:72 (2018).
Elangovan et al., Biomaterials, 35:737 (2014).
Elangovan et al., Biomaterials, 35:737 (2014).
Elangovan et al., J. Cont. Rel., 218:22 (2015).
Elangovan et al., J. Control Release, 218:22 (2015).
Elangovan et al., J. Periodo., 84:117 (2013).
Evans et al., J. Cell. Phys., 227:416 (2012).
Evans, Ex. Rev. Mol. Med., 12: ______ (2010).
Franqui-Machin et al., J. Clin. Invest., 128:2877 (2018).
Fusco et al., Sci. Signal., 9: ra96 (2016).
Giannoudis et al., Injury, 36: S20 (2005).
Gooding et al., Nat. Commun., 10:4533 (2019).
Guo et al., Cell. Death Dis., 11:847 (2020).
Hartmann & Lucius, Int. J. Parasitol., 33:1291 (2003).
Hay et al., Exp. Hematol., 68:51 (2018).
Henriksen et al., Endocr. Rev., 32:31 (2011).
Intra et al., J. Pharm. Sci., 99:368 (2010).
Jin et al., Cell Res., 22:1356 (2012).
Jono et al., Circulation, 98:1302 (1998).
Keats et al., Cancer Cell., 12:131 (2007).
Khorsand et al., AAPS J., 19:438 (2017).
Khorsand et al., Adv. Healthc. Mater., 9: e2000770 (2020).
Khorsand et al., J. Biomed. Mater. Res. B Appl. Biomater., 107:1824 (2019).
Khorsand et al., J. Control Release, 248:53 (2017).
Lack et al., J. Bone Joint Surg. Am., 95:488 (2013).
Laird ET AL., Adv. Drug Deliv. Rev., 174:613 (2021).
Laird et al., J. Tissue Eng. Regen. Med., 14:622 (2020).
Ledergor et al., Nat. Med., 24:1867 (2018).
Lee et al., Sci. Rep., 6:27336 (2016).
Lerner et al., Acta Physiol. Scand., 161:81 (1997).
Li et al., Theranostics, 11:9821 (2021).
Mohammad et al., Methods Mol. Biol., 455:37 (2008).
Morris et al., Mol. Pharm., 14:2166 (2017).
Moussa & Dym, Dental Clin., 64:473 (2020).
Na et al., Int. J. Mol. Sci., 21:8581 (2020).
Naguib et al., Drug Deliv. Transl. Res., 11:182 (2021).
Novack et al., J. Exp. Med., 198:771 (2003).
Novack, Cell Res., 21:169 (2011).

### Oortveld et al., FASEB J., 31:4286 (2017).

Panwar et al., Br. J. Pharmacol., 173:396 (2016).
Phan & Croucher, Nat. Rev. Cancer, 20:398 (2020).
Plonka et al., Gene Ther., 24:31 (2017).
Potts et al., Int. J. Exp. Pathol., 85:85 (2004).
Qiang et al., Blood, 112:196 (2008).
Rajkumar et al., Lancet Oncol., 15:e538 (2014).
Roodman, Leukemia, 23:435 (2009).
Schonemeyer et al., J. Immunol., 167:3207 (2001).
Shaughnessy et al., Blood, 109:2276 (2007).
Shaughnessy et al., Blood, 118:3512 (2011).
Shin et al., Tissue Eng, Part A, 23:1160 (2017).
Skeie et al., Cornea, 39:1285 (2020).
Song et al., J. Med. Chem., 64:2851 (2021).
Spencer et al., J. Cell. Sci., 119:1283 (2006).
Strålberg et al., FASEB J., 27:2687 (2013).
Tannoury & An, Spine J., 14:552 (2014).
Teitelbaum, Science, 289:1504 (2000).
Terpos et al., Blood, 133:1534 (2019).
Tian et al., New Eng. J. Med., 349:2483 (2003).
Tsukasaki et al., Nat. Metab., 2:1382 (2020).
Walker et al., J. Clin. Oncol., 33:3911 (2015).
Wallin et al., J. Biol. Chem., 292:14413 (2017).
Wang et al., Biomed. Pharmacother., 115:108916 (2019).
Wang et al., NanoImpact., 18: (2020).
White III, J. Bone Joint Surg. Am., 59:188 (1977).
Wongrakpanich et al., Environ. Sci. Nano., 3:365 (2016).
Woo, J. Oral Maxillof. Surg., 70:767 (2012).
Xiu et al., J. Clin. Invest., 124:297 (2014).
Xu et al., Cancer Res., 76:6901 (2016).
Yamaza et al., Bone, 29:42 (2001).
Yang et al., Blood, 122:1437 (2013).
Yeon et al., Arch. Pharm. Res., 42:712 (2019).
Zavidij et al., Nat. Cancer, 1:493 (2020).
Zeeuwen et al., FASEB J., 24:3744 (2010).
Zhan et al., Blood, 108:2020 (2006).
Zhang et al., Bioconjug. Chem., 18:2068 (2007).
Zhang et al., J. Microencapsul., 25:1 (2008).
Zhou et al., Blood, 119: e148 (2012).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising chemically modified RNA (cmRNA) encoding CST6 or a polypeptide with at least 80% amino acid sequence identity thereto.

2. The composition of claim 1 further comprising lipids.

3. The composition of claim 2 wherein the lipids form lipid particles which are encapsulated in or complexed with a biocompatible, bioresorbable scaffold.

4. The composition of claim 3 wherein the scaffold comprises a natural polymer.

5. The composition of claim 4 wherein the natural polymer comprises collagen, hyaluronic acid, chitosan, fibronectin, proteoglycan, alginate or extracellular matrix.

6. The composition of claim 2, wherein the lipids form microparticles or nanoparticles.

7. The composition of claim 6 wherein the lipid particles have a diameter of about 70 to 100 nm.

8. The composition of claim 2, wherein the cmRNA/lipid ratio is about 0.05 wt/wt to about 0.5 wt/wt.

9. The composition of claim 1, wherein the cmRNA is in an amount that inhibits osteoclast maturation, promotes osteoblast differentiation, enhances bone development, enhances fracture healing, or any combination thereof.

10. The composition of claim 1 further comprising a delivery vehicle comprising cationic or non-cationic polymers.

11. The composition of claim 10 wherein the delivery vehicle comprises PEI, poly(lactic-co-glycolic acid) (PLGA), PLLA, polystyrene, PLA, chitosan, cyclodextrin or dendrimers.

12. The composition of claim 11 wherein the dendrimer comprises polyamidoamine (PAMAM) dendrimers.

13. The composition of claim 11 wherein the delivery vehicle comprises hybrid PLGA/PEI nanoparticles.

14. The composition of claim 11 wherein the PEI comprises branched PEI.

15. The composition of claim 1, further comprising an osteogenic compound in an amount that enhances differentiation.

16. The composition of claim 1, wherein the cmRNA comprises 5-methylcytidine-5′-triphosphate, 2-thiouridine-5′-triphosphate, or combinations thereof.

17. The composition of claim 16 wherein the percent of modified nucleotides in the cmRNA is about 5% to 15%.

18. A method to enhance bone regeneration in vivo comprising administering an effective amount of the composition of claim 1 to a mammal in need thereof.

19. The method of claim 18 wherein the mammal is a human.

20. The method of claim 18, wherein the composition is administered to a bone defect in the mammal.

21. The method of claim 20 wherein the bone defect is a fracture.
